(19) United States
(12) Patent Application Publication
(10) Pub. No.: US 2021/0177952 A1
(43) Pub. Date: Jun. 17, 2021

(54) METHOD AND COMPOSITION FOR STIMULATING IMMUNE RESPONSE

(71) Applicant: BeyondSpring Pharmaceuticals, Inc., New York, NY (US)

(72) Inventors: Ramon Mohanlal, New York, NY (US); Lan Huang, New York, NY (US); James R. Tonra, New York, NY (US)

(21) Appl. No.: 17/268,380

(22) PCT Filed: Aug. 16, 2019
(86) PCT No.: PCT/US2019/046944

§ 371 Date: Feb. 12, 2021

Related U.S. Application Data
(60) Provisional application No. 62/765,099, filed on Aug. 16, 2018.

(51) Int. Cl. A61K 39/00; A61K 31/496; A61K 39/04; A61K 39/02; A61K 39/29; A61K 39/13

(52) U.S. Cl. CPC A61K 39/0011; A61K 31/496; A61K 39/04; A61K 39/107; A61K 2039/585; A61K 39/29; A61K 39/13; A61K 2039/5154; A61K 2039/54; A61K 39/0016

(57) ABSTRACT

A composition for administration to a subject is disclosed and the composition comprises a vaccine and plinabulin without or with an adjuvant to induce, enhance or boost humoral response. A method of treatment by administering a vaccine and plinabulin is disclosed. A method of enhancing an immune response to a vaccine in a subject by administering to the subject a vaccine and plinabulin is also disclosed. The vaccine and plinabulin can be administered concurrently or separately.

### CROSS-REFERENCE TO RELATED APPLICATIONS

This application is a national phase application of international PCT Application No. PCT/US2019/046944, filed on Aug. 16, 2019, which claims of U.S. Provisional Application No. 62/765,099, filed on Aug. 16, 2018, which is hereby incorporated by reference in its entirety.

### BACKGROUND

### Field

The present disclosure relates to a composition comprising a vaccine and a tubulin binding agent and method of treatment using a vaccine and a tubulin binding agent.

The human immune system comprises numerous different types of cells having overlapping functions which together act to protect the human body against sickness and disease. The cells of the immune system have complex multiple functions and interconnecting relationships. A major component of the immune system that plays an essential role in protecting the host against infection by these organisms is the humoral antibody response.

Antibodies, also known as immunoglobulins, are protein molecules which have specificity for the foreign particle which stimulates their production. Immunoglobulins (Ig) are a class of structurally related proteins consisting of two pairs of polypeptide chains. Both chains have regions that contribute to the binding of antigen and that are highly variable from one Ig molecule to another. Immunoglobulin M (IgM) is one of several forms of antibody that appear in response to initial exposure to an antigen.

The immunoglobulins derive from antibody-secreting cells. The precursors of the antibody-secreting cell are B lymphocytes, also known as “B cells.” B cells bear as a cell-surface receptor an immunoglobulin (Ig) molecule specialized for expression on the cell surface. Newly differentiated B cells initially express surface Ig solely of the IgM class. Associated with maturation of a B cell is the appearance of other immunoglobulin isotypes on the surface of the B cell. There are various ways to activate B cells, including cross-linkage of membrane (m) Ig molecules by the antigen mIg (cross-linkage-dependent B cell activation), direct encounter with T cells (helper T cells or helper T cell-associated molecules, such as, for example, CD40 ligand), or encounter with mitogens. In such encounters, the antigen presents epitopes recognized by the B cell's cell-surface Ig.

### SUMMARY

Some embodiments relate to a composition for administration to a subject, comprising a vaccine, and a tubulin binding agent. Some embodiments relate to a composition for administration to a subject, comprising a vaccine, and plinabulin.

Some embodiments relate to a method of treatment, the method comprising administering to the subject a vaccine and a tubulin binding agent. Some embodiments relate to a method of treatment, the method comprising administering to the subject a vaccine and plinabulin.

Some embodiments relate to a method of enhancing an immune response to a vaccine in a subject, said method comprising administering to the subject a vaccine and a tubulin binding agent, wherein the immune response to the vaccine is enhanced compared to the immune response generated by administration of the vaccine alone, without the tubulin binding agent, to the subject. Some embodiments relate to a method of enhancing an immune response to a vaccine in a subject, said method comprising administering to the subject a vaccine and plinabulin, wherein the immune response to the vaccine is enhanced compared to the immune response generated by administration of the vaccine alone, without plinabulin, to the subject.

Some embodiments relate to a method of inducing lymphocyte cell proliferation, comprising administering an effective amount of a tubulin binding agent and a vaccine to a subject in need thereof. Some embodiments relate to a method of inducing lymphocyte cell proliferation, comprising administering an effective amount of plinabulin and a vaccine to a subject in need thereof.

Some embodiments relate to a method of inducing B cell proliferation, comprising administering an effective amount of a tubulin binding agent and a vaccine to a subject in need thereof. Some embodiments relate to a method of inducing B cell proliferation, comprising administering an effective amount of plinabulin and a vaccine to a subject in need thereof.

Some embodiments relate to a method of inducing a production of one or more immunoglobulin, comprising administering an effective amount of a tubulin binding agent and a vaccine to a subject in need thereof. Some embodiments relate to a method of inducing a production of one or more immunoglobulin, comprising administering an effective amount of plinabulin and a vaccine to a subject in need thereof. In some embodiments, the immunoglobulin is selected from the group consisting of IgG, IgM, IgA, IgD, and IgE.

Some embodiments relate to a composition for administration to a subject, comprising an antigen or immunogen associated with an infectious disease or cancer and a tubulin binding agent. Some embodiments relate to a composition for administration to a subject, comprising an antigen or immunogen associated with an infectious disease or cancer and plinabulin.

Some embodiments relate to a method of treatment, the method comprising administering to the subject an antigen or immunogen associated with an infection disease or cancer and a tubulin binding agent. Some embodiments relate to a method of treatment, the method comprising administering to the subject an antigen or immunogen associated with an infection disease or cancer and plinabulin.

Some embodiments relate to a composition for administration to a subject, comprising an antigen or immunogen presenting cell based vaccine and a tubulin binding agent. Some embodiments relate to a composition for administration to a subject, comprising an antigen or immunogen presenting cell based vaccine and plinabulin.

Some embodiments relate to a method of treatment, the method comprising administering to the subject a vaccine comprising an antigen or immunogen presenting cell based vaccine and a tubulin binding agent. Some embodiments relate to a method of treatment, the method comprising administering to the subject a vaccine comprising an antigen or immunogen presenting cell based vaccine and plinabulin.

Some embodiments relate to a composition for administration to a subject, comprising a dendritic cell based vaccine and a tubulin binding agent. Some embodiments relate to a composition for administration to a subject, comprising a dendritic cell based vaccine and plinabulin.

Some embodiments relate to a method of treatment, the method comprising administering to the subject a dendritic cell based vaccine and a tubulin binding agent. Some embodiments relate to a method of treatment, the method comprising administering to the subject a dendritic cell based vaccine and plinabulin.

Some embodiments relate to a composition for administration to a subject, comprising a B cell based vaccine and a tubulin binding agent. Some embodiments relate to a composition for administration to a subject, comprising a B cell based vaccine and plinabulin.

Some embodiments relate to a method of treatment, the method comprising administering to the subject a B cell based vaccine and a tubulin binding agent. Some embodiments relate to a method of treatment, the method comprising administering to the subject a B cell based vaccine and plinabulin.

Some embodiments relate to a method of enhancing immune response, the method comprising administering a vaccine and a tubulin binding agent, wherein the tubulin binding agent is administered after the administration of vaccine. In some embodiments, the tubulin binding agent is plinabulin.

Some embodiments relate to a method of treatment, the method comprising administering a vaccine and a tubulin binding agent, wherein the a tubulin binding agent is administered after the administration of vaccine.

### DESCRIPTION OF DRAWINGS

FIGS. 1A to 4J illustrate the enhancing effects of tubulin-binding agents (e.g., plinabulin) on B-cell response to ovalbumin immunization as exemplified in the study as described in Example 2.

FIGS. 1A-1E illustrate changes of average body weight of mice over the study of Example 2 in five subgroups: Subgroup 1 (FIG. 1A), Subgroup 2 (FIG. 1B), Subgroup 3 (FIG. 1C), Subgroup 4 (FIG. 1D), Subgroup 5 (FIG. 1E).

FIG. 2 illustrates changes of average body weight of mice between Day 1 and Day 62 in the study of Example 2. The error bars show the standard deviations.

FIGS. 3A-3F illustrate individual mouse serum level of ovalbumin IgG1 (ng/mL) on Day 30 (FIGS. 3A, 3C, and 3E) and Day 62 (FIGS. 3B, 3D, and 3F) immunization in the study of Example 2. Animals 3501, 3502, 3503, 3504, and 3505, shown in FIGS. 3E-3F, received 15 mg/kg instead.

FIGS. 4A-4J illustrate serum level of ovalbumin IgG1 in Subgroups 1-5 on Day 30 (FIGS. 4A, 4C, 4E, 4G, and 4I) and Day 62 (FIGS. 4B, 4D, 4F, 4H, and 4J) in the study of Example 2. The plinabulin was administered BID in a single day at a specified time after immunization: 1 hour post-immunization (FIGS. 4A-4B), Day 3 (FIGS. 4C-4D), Day 6 (FIGS. 4E-4F), Day 14 (FIGS. 4G-4H), and Day 28 (FIGS. 41-4J). The symbols “*” and “**” indicate, respectively, p<0.05 and p<0.01, as compared with the corresponding vehicle group. The error bars show the standard deviations.

FIGS. 5 through 8 illustrate the enhancing effects of tubulin-binding agents (e.g., plinabulin) on CD4⁺ T-cell response induced by CD14⁺ dendritic cells as exemplified in the study as described in Example 3A.

FIG. 5 illustrates FACS (Fluorescence-Activated Cell Sorting) profiles of dendritic cells treated with plinabulin during differentiation in Study Arm #A1. FIG. 6 illustrates FACS profiles of dendritic cells treated with plinabulin during maturation in Study Arm #A2. FIG. 7 illustrates effect of test article on IL-2 secretion in MLR (Mixed Lymphocyte Reaction) in the study as described in Example 3A. FIG. 8 illustrates effect of test article on IFN-y secretion in MLR in the study of Example 3A. In FIGS. 7-8, the data of Study Arm #s A1-A3 were combined and plotted.

FIGS. 9 through 12 illustrate the enhancing effects of tubulin-binding agents (e.g., plinabulin) on CD4⁺ T-cell response induced by CD14⁺ dendritic cells as exemplified in the study as described in Example 3B.

FIG. 9 illustrates FACS profiles of dendritic cells treated with plinabulin during differentiation in Study Arm #B1. FIG. 10 illustrates FACS profiles of dendritic cells treated with plinabulin during maturation in Study Arm #B2. FIG. 11 illustrates effect of test article on IL-2 secretion in MLR in the study of Example 3B. FIG. 12 illustrates effect of test article on IFN-γ secretion in MLR in the study, as described in Example 3B. In FIGS. 11-12, the data of Study Arm #s B 1, B2, and B3 were combined and plotted as mean +/−SEM.

FIGS. 13 and 14 illustrate the enhancing effects of tubulin-binding agents (e.g., plinabulin) on CD4⁺ T-cell response induced by CD14⁺ dendritic cells as exemplified in the study as described in Example 3C.

FIG. 13 illustrates effect of test article on IL-2 secretion; and FIG. 14 illustrates effect of test article on IFN-γ secretion in MLR assay in the study of Example 3C. The data in FIGS. 13-14 were plotted as mean +/−SEM.

### DETAILED DESCRIPTION

The immune system encompasses cellular immunity and humoral immunity. Cellular immunity includes a network of cells and events. Humoral immunity involves B cells and antibodies. When B cells become transformed to plasma cells, the plasma cells express and secrete antibodies. The secreted antibodies can subsequently bind to antigens residing on the surface of infected or tumor cells. The result is that the infected cells or tumor cells become tagged with the antibody. With binding of the antibody to the infected cell or tumor cell, the bound antibody mediates killing of the infected cell or tumor cell.

Plinabulin, (3Z,6Z)-3-Benzylidene-6-{[5-(2-methyl-2-propanyl)-1H-imidazol-4-yl]methylene}-2,5-piperazinedione, is a synthetic analog of the natural compound phenylahistin. Plinabulin can be readily prepared according to methods and procedures detailed in U.S. Pat. Nos. 7,064,201 and 7,919,497, which are incorporated herein by reference in their entireties.

Plinabulin can be effective in activating B cell, inducing B cell proliferation and maturation, and further inducing (through dedicated plasma cells) Immunoglobulin (e.g, IgG, IgM, IgA, IgD, and IgE) antibody production and secretion that is specific to the presented antigen.

Some embodiments relate to the use of a tubulin binding agent in combination with one or more vaccines for treatment or enhancing the immune response in a subject. Some embodiments relate to the use of Plinabulin in combination with one or more vaccines for treatment or enhancing the immune response in a subject.

Administration of a vaccine and a tubulin binding agent such as plinabulin can increase the intensity, rate, and duration of immune response, and/or shorten onset time of antibody responses. The immune response can be humoral immune response. The combination of a vaccine and plinabulin can also increase the number of antibody producing B cells, increase the rate at which neutralizing antibodies such as IgG, IgM, IgA, IgD, and IgE are produced, extend the duration for which antibodies are generated, and/or shorten the onset time, as compared to the administration of the vaccine alone. Therefore, using a vaccine and plinabulin can stimulate greater protection against the pathological and/or immunogenic targets expressing the antigen in the vaccine. In addition, using a vaccine together with plinabulin could lead to enhanced clonal expansion and memory and a quicker/faster, more intense, and more prolonged humoral response upon re-challenge to the antigen in question. The combination of vaccine and plinabulin can produce a synergistic effect and achieve greater benefit than using vaccine alone; and the combination also allows for possible use of smaller doses of the vaccine to achieve protective antibody titers.

When a tubulin binding agent is used to boost the immune response induced by the vaccine, the timing of administration can be critical. It is unexpected that administering a tubulin binding agent such as plinabulin after the vaccine administration, particularly at the time or shortly after the lymphocyte such as T cell is activated by contacting the antigen presenting cell, can greatly increase the lymphocyte expansion or proliferation and promote a stronger immune response than administering the tubulin binding agent prior to or concurrently with vaccine. Addition of a tubulin binding agent during or shortly after the activation of lymphocyte cell can increase the T cell proliferation more effectively than the addition of the tubulin binding agent before the administration of vaccine, thus providing enhanced immune response and better protection against the immunogenic targets expressing the antigen in the vaccine.

### Definitions

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The term “inducing and/or enhancing an immune response” means that the method evokes and/or enhances any response of the animal's immune system. “Immune response” is defined as any response of the immune system, for example, of either a cell-mediated (i.e. cytotoxic T-lymphocyte mediated) or humoral (i.e. antibody mediated) nature. These immune responses can be assessed by a number of in vivo or in vitro assays well known to one skilled in the art including, but not limited to, antibody assays (for example ELISA assays) antigen specific cytotoxicity assays, production of cytokines (for example ELISPOT assays), etc.

The term “lymphatic site” means a site in the body that is associated with the lymphatic system including lymphatic organs, tissues, cells, nodes or glands such as spleen, thymus, tonsils, Peyer's patches, bone marrow, lymphocytes, thoracic duct as well as all of the lymph nodes of the body.

The term “Immunoglobulin” or “Ig” refers to a protein or antibody produced by plasma cells that is used by the immune system to neutralize antigens. There are 5 classes of human antibodies: IgG, IgM, IgA, IgD, and IgE. Some immunoglobulins, such as IgG, IgD, and IgE, are “Y”-shaped macromolecules called monomers composed of four glycoprotein chains. There are two identical heavy chains having a high molecular weight that varies with the class of antibody. In addition, there are two identical light chains of one of two varieties: kappa or gamma. Depending on the class of antibody, biological activities of the Fc portion of antibodies include the ability to activate the complement pathway (IgG & IgM), bind to phagocytes (IgG, IgA), or bind to mast cells and basophils (IgE). Some classes of immunoglobulins are more complex: for example, IgM is a pentamer, consisting of 5 “Y”-like molecules connected at their Fc portions, and secretory IgA is a dimer consisting of 2 “Y”-like molecules.

As used herein, common pharmacy abbreviations are defined as follows:
API Active Pharmaceutical Ingredient
BCG Bacillus Calmette-Guérin
BID Twice Daily (“bis in die”)
CFA Complete Freund's Adjuvant
CTG CellTiter-Glo
D5W 5% Dextrose in Water
DMSO Dimethyl Sulfoxide
ELISA Enzyme-linked immunosorbent assay
FBS Fetal Bovine Serum
g Gram(s)
G Gauge
hr(s) Hour(s)
HRP Horseradish Peroxidase
IFN-γ Interferon-γ
IgE Immunoglobulin E
IgG Immunoglobulin G
IL-2 Interleukin-2
IP Intraperitoneal
kDa Kilo-Dalton(s)
L Liter(s)
mg Microgram(s)
MLR Mixed Lymphocyte Reaction
N/A Not Applicable
OVA Ovalbumin
PBS Phosphate Buffered Saline
PG Propylene glycol
RPM Rotation Per Minute
SC Subcutaneous
SOPs Standard Operating Procedures
TMB Tetramethylbenzidine
v/v Volume/Volume
μL Microliter(s)

### Vaccine, Pharmaceutical Composition and Administration

Some embodiments relate to a composition for administration to a subject, including a vaccine and a tubulin binding agent. Some embodiments relate to a composition for administration to a subject, including a vaccine and plinabulin. In some embodiments, the composition does not include an adjuvant. In some embodiments, the composition further comprises an adjuvant to induce, enhance or boost humoral response.

In some embodiments, the vaccine can be a commercially available vaccine. In some embodiments, a commercially available vaccine can comprise at least one additional adjuvant, e.g., alum.

In some embodiments, the vaccine is selected from the vaccine against one or more diseases selected from the group consisting of cholera, dengue, diphtheria, Hoemophilus influzenzoe type b infection, hepatitis A, hepatitis B, influenza, Japanese encephalitis, meningococcal meningitis, pertussis, polio, rabies, tetanus, tuberculosis, typhoid, and yellow fever.

In some embodiments, the vaccine can be selected from the group consisting of Haemophilus b Conjugate Vaccine (Tetanus Toxoid Conjugate); Tetanus Toxoid, Reduced Diphtheria Toxoid and Acellular Pertussis Vaccine Adsorbed; Diphtheria and Tetanus Toxoids and Acellular Pertussis Vaccine Adsorbed; Diphtheria and Tetanus Toxoids Adsorbed; Quadrivalent Influenza Vaccine; High-Dose Influenza Vaccine; Quadrivalent Influenza Vaccine; Intradermal Quadrivalent Influenza Vaccine; Rabies Vaccine (Human Diploid Cell); Poliovirus Vaccine Inactivated; Meningococcal (Groups A, C, Y and W-135) Polysaccharide Diphtheria Toxoid Conjugate Vaccine; Diphtheria and Tetanus Toxoids and Acellular Pertussis Adsorbed, Inactivated Poliovirus and Haemophilus b Conjugate (Tetanus Toxoid Conjugate) Vaccine; Diphtheria and Tetanus Toxoids and Acellular Pertussis Absorbed and Inactivated Poliovirus Vaccine; Tetanus and Diphtheria Toxoids Adsorbed; Typhoid Vi Polysaccharide Vaccine; Yellow Fever Vaccine; Rabies—HT Rabies Immune Globulin (Human) USP, Heat Treated; Tuberculin Purified Protein Derivative, Mantoux; and Yellow Fever Vaccine.

The antigens or immunogens used to prepare the vaccines may be derived from a wide variety of sources. For example, suitable antigens or immunogens may include an infectious agent (e.g., bacterial, fungal, protozoan, parasitic, or viral), an infectious agent-derived product, e.g., protein, peptide, nucleic acid, polysaccharide, glycoprotein, glycolipid, antigen or antigenic preparations, a degenerative disease antigen, an atopic disease antigen, an autoimmune disease antigen, an alloantigen, a xenoantigen, a metabolic disease enzyme or enzymatic product, a recombinantly produced protein or peptide, a chimeric fusion protein, and/or a small molecule.

Suitable antigens or immunogens may be in the form of whole cells or purified or partially purified antigens or antigenic preparations. Suitable antigens or immunogens may be used without modification, in galenic form, or in combination with vehicles or carriers such as e.g. microspheres, liposomes, nanospheres, and other antigen delivery systems familiar to one of ordinary skill in the art.

The vaccine can be based on an antigen that is prepared or derived from natural sources or produced through recombinant technologies.

### Infectious Disease Vaccine

In some embodiments, the vaccine can be a vaccine for an infectious diseases. In some embodiments, the vaccine for an infectious diseases comprises an antigen or immunogen selected from microbial structures (cell walls, capsules, flagella, pili, viral capsids, envelope-associated glycoproteins); microbial toxins (Allergens: dust, pollen, hair, foods, dander, bee venom, drugs, and other agents causing allergic reactions; Foreign tissues and cells (from transplants and transfusions); and the body's own cells that the body fails to recognize as “normal self” (cancer cells, infected cells, cells involved in autoimmune diseases).

In one embodiment, the antigen or immunogen can be an infectious agent, or a product of an infectious agent. In one embodiment, the antigen or immunogen comprises an inactivated infectious agent, e.g., that has been killed or otherwise attenuated. In another embodiment, the antigen or immunogen comprises a live infectious agent.

In one embodiment, the infectious agent (or infectious agent product) is a virus, for example and without limitation, a pox virus (e.g., vaccinia virus), smallpox virus, marburg virus, flaviviruses (e.g. Yellow Fever Virus, Dengue Virus, Tick-borne encephalitis virus, Japanese Encephalitis Virus), influenza virus (or antigens, such as F and G proteins or derivatives thereof), e.g., influenza A; or purified or recombinant proteins thereof, such as HA, NP, NA, or M proteins, or combinations thereof), parainfluenza virus (e.g., sendai virus), respiratory syncytial virus, rubeola virus, human immunodeficiency virus (or antigens, e.g., such as tat, nef, gp120 or gp160), human papillomavirus (or antigens, such as HPV6, 11, 16, 18), varicella-zoster virus (or antigens such as gpl, II and IE63), herpes simplex virus (e.g., herpes simplex virus I, herpes simplex virus II; or antigens, e.g., such as gD or derivatives thereof or Immediate Early protein such as ICP27 from HSV1 or HSV2), cytomegalovirus (or antigens such as gB or derivatives thereof), Epstein-Barr virus (or antigens, such as gp350 or derivatives thereof), JC virus, rhabdovirus, rotavirus, rhinovirus, adenovirus, papillomavirus, parvovirus, picomavirus, poliovirus, virus that causes mumps, virus that causes rabies, reovirus, rubella virus, togavirus, orthomyxovirus, retrovirus, hepadnavirus, hantavirus, junin virion, filovirus (e.g., ebola virus), coxsackievirus, equine encephalitis virus, Rift Valley fever virus, alphavirus (e.g., Chikungunyavirus, sindbis virus), hepatitis A virus, hepatitis B virus (or antigens thereof, for example Hepatitis B Surface antigen or a derivative thereof), hepatitis C virus, hepatitis D virus, or hepatitis E virus.

In one embodiment, the infectious agent is a bacterium. Non-limiting examples of suitable bacteria (or bacterially derived products) for use in the vaccines and/or methods of the invention include Neisseria species, including N. gonorrhea and N. meningitidis (or antigens, such as, for example, capsular polysaccharides and conjugates thereof, transferrin-binding proteins, lactoferrin binding proteins, Pi1C, adhesins); Haemophilus species, e.g., H. influenzae; S. pyogenes (or antigens, such as, for example, M proteins or fragments thereof, C5A protease, lipoteichoic acids), S. agalactiae, S. mutans; H. ducreyi; Moraxella spp, including M catarrhalis, also known as Branhamella catarrhalis (or antigens, such as, for example, high and low molecular weight adhesins and invasins); Bordetella spp, including B. pertussis (or antigens, such as, for example, pertactin, pertussis toxin or derivatives thereof, filamenteous hemagglutinin, adenylate cyclase, fimbriae), B. parapertussis and B. bronchiseptica; Mycobacterium species, including M. tuberculosis (or antigens, such as, for example, ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (or antigens, such as, for example, colonization factors, heat-labile toxin or derivatives thereof, heat-stable toxin or derivatives thereof), enterohemorragic E. coli, enteropathogenic E. coli (or antigens, such as, for example, shiga toxin-like toxin or derivatives thereof); Vibrio spp, including V. cholera (or antigens, such as, for example, cholera toxin or derivatives thereof); Shigella spp, including S. sonnei, S. dysenteriae, S. flexnerii; Yersinia spp, including Y enterocolitica (or antigens, such as, for example, a Yop protein), Y pestis, Y. pseudotuberculosis; Campylobacter spp, including C. jejuni (or antigens, such as, for example, toxins, adhesins and invasins) and C. coli; Salmonella spp, including S. typhi, S. paratyphi, S. choleraesuis, S. enteritidis, S. typhimurium, and S. dysenteriae; Listeria species, including L. monocytogenes; Helicobacter spp, including H. pylori (for example urease, catalase, vacuolating toxin); Pseudomonas spp, including P. aeruginosa; Staphylococcus species, including S. aureus, S. epidermidis; Proteus species, e.g., P. mirabilis; Enterococcus species, including E. faecalis, E. faecium; Clostridium species, including C. tetani (or antigens, such as, for example, tetanus toxin and derivative thereof), C. botulinum (or antigens, such as, for example, botulinum toxin and derivative thereof), C. difficile (or antigens, such as, for example, Clostridium toxins A or B and derivatives thereof), and C. perfringens; Bacillus species, including B. anthracis (or antigens, such as, for example, botulinum toxin and derivatives thereof), B. cereus, B. circulans and B. megaterium; Corynebacterium species, including C. diphtheriae (or antigens, such as, for example, diphtheria toxin and derivatives thereof); Borrelia species, including B. burgdorferi (for example OspA, OspC, DbpA, DbpB), B. garinii (or antigens, such as, for example, OspA, OspC, DbpA, DbpB), B. afzelii (for example OspA, OspC, DbpA, DbpB), B. andersonii (or antigens, such as, for example, OspA, OspC, DbpA, DbpB), B. hermsii; Ehrlichia species, including E. equi and the agent of the Human Granulocytic Ehrlichiosis; Rickettsia spp, including R. rickettsii; Chlamydia species, including C. trachomatis (or antigens, such as, for example, MOMP, heparin-binding proteins), C. pneumoniae (for example MOMP, heparin-binding proteins), C. psittaci; Leptospira species, including L. interrogans; Streptococcus species, such as S. pyogenes, S. agalactiae, S. pneumonia; Treponema species, including T. pallidum (or antigens, such as, for example, the rare outer membrane proteins), T denticola, and T. hyodysenteriae.

In one embodiment, the infectious agent is a parasite, or a parasite derived product. Non-limiting examples of suitable parasite (or parasite derived products) for use in the vaccines and/or methods of the invention include Plasmodium species, including P. falciparum; Toxoplasma species, including T. gondii (or antigens, such as, for example SAG2, SAGS, Tg34); Entamoeba species, including E. histolytica; Babesia species, including B. microti; Trypanosoma species, including T cruzi; Giardia species, including G. lamblia; Leshmania species, including L. major; Pneumocystis species, including P. carinii; Trichomonas species, including T. vaginalis; and Schisostoma species, including S. mansoni.

In another embodiment, the infectious agent is a fungus, or a fungal derived product. Suitable fungi (or fungal derived products) for use in the vaccines and/or methods of the invention include, without limitation, Candida species, including C. albicans and parapsilosis; Cryptococcus species, including C. neoformans; Aspergillus fumigates and niger, Fusarium spp, Trychophyton spp, Absidia species, e.g., Absidia corymbifera, Ajellomyces spp, e.g., Ajellomyces capsulatus, Arthroderma species, e.g., Arthroderma benhamiae, Blastomyces species, e.g., Blastomyces dermatitidis, Cladophialophora species, e.g., Cladophialophora carrionii, Coccidioides spp, e.g., Coccidioides immitis, Cryptococcus spp, e.g., Cryptococcus neoformans, Cunninghamella species, Epidermophyton species, e.g., Epidermophyton floccosum, Exophiala spp, e.g., Exophiala dermatitidis, Filobasidiella spp, e.g., Filobasidiella neoformans, Fonsecaea spp, e.g., Fonsecaea pedrosoi, Fusarium spp, e.g., Fusarium solani, Geotrichum spp, e.g., Geotrichum candidum, Histoplasma spp, e.g., Histoplasma capsulatum, Hortaea spp, e.g., Hortaea werneckii, Issatschenkia spp, e.g., Issatschenkia orientalis, Madurella spp, e.g., Madurella grisae, Malassezia spp, e.g., Malassezia furfur, Microsporum spp, e.g., Microsporum canis, Mucor spp, e.g., Mucor circinelloides, Nectria spp, e.g., Nectria haematococca, Paecilomyces spp, e.g., Paecilomyces variotii, Paracoccidioides spp, e.g., Paracoccidioides brasiliensis, Penicillium spp, e.g., Penicillium marneffei, Pichia spp, e.g., Pichia guilliermondii, Pneumocystis spp, e.g., Pneumocystis carinii, Pseudallescheria spp, e.g., Pseudallescheria boydii, Rhizopus spp, e.g., Rhizopus oryzae, Rhodotorula spp, e.g., Rhodotorula rubra, Scedosporium spp, e.g., Scedosporium apiospermum, Schizophyllum spp, e.g., Schizophyllum commune, Sporothrix spp, e.g., Sporothrix schenckii, Trichophyton spp, e.g., Trichophyton violaceum, and Trichosporon spp, e.g., Trichosporon mucoides.

In another embodiment, the infectious agent is a protozoan, or a protozoan derived product. Suitable protozoans (or protozoan derived products) for use in the vaccines and/or methods of the invention include, without limitation, protests (unicellular or multicellular), e.g., Plasmodium falciparum, and helminths, e.g., cestodes, nematodes, and trematodes.

In one embodiment, a suitable antigen or immunogen for use in the vaccines and methods of the invention is an alloantigen (a self-antigen), such as a protein or peptide, lipoprotein, lipid, carbohydrate, a nucleic acid, an enzyme, a structural protein, a secreted protein, a cell surface receptor, and a cytokine, e.g., TNF, IFN-γ, IL-1, or IL-6. In one embodiment, the self-antigen is cholesteryl ester transfer protein (CETP), the Aβ protein associated with Alzheimer's, a proteolytic enzyme that processes the pathological form of the Aβ protein, e.g., beta-secretase, LDL associated with atherosclerosis, or a coreceptor for HIV-I, e.g., CCR5. In one embodiment, the LDL associated with atherosclerosis is oxidized or minimally modified.

### Cancer Vaccine

In some embodiments, the vaccine can be a cancer vaccine. The cancer vaccine can comprise an antigen or immunogen capable of activating the immune response. The cancer vaccine can also include any DNA damaging agents. Administration of plinabulin in combination with a cancer vaccine can stimulate greater protection against the pathological and/or immunogenic targets expressing the antigen or immunogen in the vaccine, quicker/faster, and/or more intense, and/or for a longer period of time, as compared to using vaccine alone. A tubulin binding agent such as plinabulin, when used in combination with a cancer vaccine, can lead to a more effective immune response to delay or stop cancer cell growth; to cause tumor shrinkage; to prevent cancer from coming back; or to eliminate cancer cells that have not been killed by other forms of treatment.

The DNA damaging agents include exogenous sources of agents that can cause DNA damages in cells or inhibit the repair of endogenous DNA damage in cells. In some embodiments, the DNA damaging agents can increase antigen presentation and promote immune response to cancer cells. In some embodiments, the DNA damaging agents can include a chemotherapy and/or radiation therapy. In some embodiments, the DNA damaging agent can include alkylating agents (e.g., cyclophosphamide and ifosfamide), platinum based compounds (e.g. cisplatin, carboplatin and oxaliplatin), antimetabolites (e.g., gemcitabine, methotrexate and pemetrexed), anthrycyclines (e.g. doxorubicin and epirubicin), topoisomerase I inhibitors (e.g., etoposide), topoisomerase II inhibitors (e.g. irinotecan and topotecan), radiomimetics (e.g. bleomycin) and other anti-mitotics (e.g. docetaxel, paclitaxel and vinorelbine). The DNA damaging agent described herein can create new foreign epitope in cancer cells that the immune system can recognize and mount an immune response towards, thus functioning as a vaccine and leading to an anti-cancer immune response. A tubulin binding agent such as plinabulin, when used in combination with a cancer vaccine such as a DNA damaging agent, can lead to an enhanced anti-tumor immune response and more effective killing of cancer cells.

In some embodiments, cancer vaccine may be made from a patient's own tumor cells (that is, they are customized so that they mount an immune response against features that are unique to a specific patient's tumor). In some embodiments, cancer vaccine may be made from substances (antigens or immunogens) that are produced by certain types of tumors (that is, they mount an immune response in any patient whose tumor produces the antigen or immunogen).

In some embodiments, the cancer vaccine comprises cancer antigen or cancer immunogen that is substantially loaded into dendritic cell (DC), antigen presenting cell (APC) or B-cell. The first FDA-approved cancer treatment vaccine, sipuleucel-T is created by isolating immune system cells called dendritic cells, which are a type of antigen-presenting cell (APC), from a patient's blood. These cells are sent to the vaccine manufacturer, where they are cultured in the laboratory together with a protein called PAP-GM-CSF. This protein consists of PAP linked to a protein called granulocyte-macrophage colony-stimulating factor (GM-CSF), which stimulates the immune system and enhances antigen presentation.

Several strategies have been used to load dendritic cell (DC), antigen presenting cell (APC), or B cell with cancer antigen: 1) Synthetic peptide or purified proteins can be pulsed onto the DC, APC, or B cell surface. 2) DC, APC, or B cell can be engineered with plasmid DNA, RNA, or viruses to express specific gene products. 3) Tumor lysate, tumor RNA, tumor cell lysates, and auto phagosomes can be mixed with APC or immature DC or B cell so that the APC or DC will process and present multiple peptides. 4) DC or APC or B cell can be fused with entire tumor cells via PEG or electroporation.

In some embodiments, the cancer vaccine is an APC based vaccine. In some embodiments, the cancer vaccine is a DC based vaccine. In some embodiments, the cancer vaccine is a B cell based vaccine. In some embodiments, the cancer vaccine does not comprise a check point inhibitor.

Some examples of cancer vaccine include but are not limited to sipuleucel-T, Trastuzumab, rituximab, ofatumumab, alemtuzumab, antibody-drug conjugates (ADCs) such as ado-trastuzumab emtansine, brentuximab vedotin; blinatumomab, denileukin diftitox, or talimogene laherparepvec.

In one embodiment, the cancer vaccine can comprise a “self”-antigen that is a tumor-associated antigen.

In some embodiments, the cancer vaccine and the tubulin binding agent (e.g., plinabulin) are administered without an adjuvant to induce, enhance or boost humoral response. In some embodiments, the cancer vaccine and and the tubulin binding agent (e.g., plinabulin) are administered with an adjuvant to induce, enhance or boost humoral response.

Antigens or immunogens suitable for use in the vaccines and methods of the invention may be obtained from any source. For example, infectious agents for use in formulating the vaccines of the present invention can be obtained from commercial sources, including, but not limited to, American Type Culture Collection (ATCC). In some embodiments, the infectious agents are passed in cell culture and/or animals prior to being combined with a bisphosphonate and a pharmaceutically acceptable carrier. In other embodiments, suitable antigens or immunogens not purified (or cellular lysates), partially purified (e.g., cell lysates have been removed), or purified. In other embodiments, suitable antigens are prepared recombinantly.

In one embodiment, a suitable antigen or immunogen is present in a commercially available vaccine (e.g., a commercially available vaccine comprising alum). In one embodiment, the commercially available vaccine for use in the compositions and methods described herein has been approved by a regulatory agency such as, for example, the United States Food and Drug Administration, the European Medicines Agency (EMA), the Japanese Ministry of Health and Welfare (MHW), the Therapeutic Goods Administration of Australia, the State Food and Drug Administration (SFDA) (China), and the Health Protection Branch of Canada.

The commercially suitable vaccines suitable for use in the compositions and methods described herein include, for example, vaccines suitable for human and veterinary administration.

Examples of commercially available vaccines for use in the vaccines and methods of the invention include, without limitation, those listed below in Table A.

TABLE A
Non-Limiting Examples of Commercially-Available Vaccines

| Vaccine Name | Trade name | Company |
|---|---|---|
| Adenovirus Type 4 and Type 7 Vaccine, Live, | N/A | Barr Labs, Inc. |
| Oral | | |
| Anthrax Vaccine Adsorbed | Biothrax | Emergent BioDefense Operations |
| | | Lansing Inc. |
| BCG Live | BCG Vaccine | Organon Teknika Corp LLC |
| BCG Live | Mycobax | Sanofi Pasteur, Ltd |
| BCG Live | TICE BCG | Organon Teknika Corp LLC |
| Diphtheria & Tetanus Toxoids Adsorbed | No Trade Name | Sanofi Pasteur, Inc |
| Diphtheria & Tetanus Toxoids Adsorbed | No Trade Name | Sanofi Pasteur, Ltd |
| Diphtheria & Tetanus Toxoids & | Tripedia | Sanofi Pasteur, Inc |
| Acellular Pertussis Vaccine Adsorbed | | |
| Diphtheria & Tetanus Toxoids & | Infanrix | GlaxoSmithKline Biologicals |
| Acellular Pertussis Vaccine Adsorbed | | |
| Diphtheria & Tetanus Toxoids & | DAPTACEL | Sanofi Pasteur, Ltd |
| Acellular Pertussis Vaccine Adsorbed | | |
| Diphtheria & Tetanus Toxoids & Acellular | Pediarix | GlaxoSmithKline Biologicals |
| Pertussis Vaccine Adsorbed, Hepatitis B | | |
| (recombinant) and Inactivated Poliovirus | | |
| Vaccine Combined | | |
| Diphtheria and Tetanus Toxoids and Acellular | KINRIX | GlaxoSmithKline Biologicals |
| Pertussis Adsorbed and Inactivated Poliovirus | | |
| VaccinelO | | |
| Diphtheria and Tetanus Toxoids and Acellular | Pentacel | Sanofi Pasteur Limited |
| Pertussis Adsorbed, Inactivated Poliovirus and | | |
| Haemophilus b Conjugate (Tetanus Toxoid | | |
| Conjugate) Vaccine | | |
| Haemophilus b Conjugate Vaccine | PedvaxHIB | Merck & Co, Inc |
| (Meningococcal Protein Conjugate) | | |
| Haemophilus b Conjugate Vaccine (Tetanus | ActHIB | Sanofi Pasteur, SA |
| Toxoid Conjugate) | | |
| Haemophilus b Conjugate Vaccine (Tetanus | Hiberix | GlaxoSmithKline Biologicals, S.A. |
| Toxoid Conjugate) | | |
| Haemophilus b Conjugate Vaccine | Comvax | Merck & Co, Inc |
| (Meningococcal Protein Conjugate) & | | |
| Hepatitis B Vaccine (Recombinant) | | |
| Hepatitis A Vaccine, Inactivated | Havrix | GlaxoSmithKline Biologicals |
| Hepatitis A Vaccine, Inactivated | VAQTA | Merck & Co, Inc |
| Hepatitis A Inactivated and Hepatitis B | Twinrix | GlaxoSmithKline Biologicals |
| (Recombinant) Vaccine | | |
| Hepatitis B Vaccine (Recombinant) | Recombivax HB | Merck & Co, Inc |
| Hepatitis B Vaccine (Recombinant) | Engerix-B | GlaxoSmithKline Biologicals |
| Human Papillomavirus Quadrivalent (Types | Gardasil | Merck and Co, Inc. |
| 6, 11, 16, 18) Vaccine, Recombinant | | |
| Human Papillomavirus Bivalent (Types 16, | Cervarix | GlaxoSmithKline Biologicals |
| 18) Vaccine, Recombinant | | |
| Influenza A (H1N 1) 2009 Monovalent Vaccine | No Trade Name | CSL Limited |
| Influenza A (H1N 1) 2009 Monovalent Vaccine | No Trade Name | Medimmune LLC |
| Influenza A (H1N 1) 2009 Monovalent Vaccine | No Trade Name | ID Biomedical Corporation of |
| | | Quebec |
| Influenza A (H1N 1) 2009 Monovalent Vaccine | No Trade Name | Novartis Vaccines and Diagnostics |
| | | Limited |
| Influenza A (H1N 1) 2009 Monovalent Vaccine | No Trade Name | Sanofi Pasteur, Inc. |
| Influenza Virus Vaccine | Afluria | CSL Limited |
| Influenza Virus Vaccine, H5N 1 (for National | No Trade Name | Sanofi Pasteur, Inc. |
| Stockpile) | | |
| Influenza Vims Vaccine, Trivalent, Types A | FluLaval | ID Biomedical Corp of Quebec |
| and B | | |
| Influenza Vaccine, Live, Intranasal | FluMist | Medimmune, LLC |
| Influenza Vims Vaccine, Trivalent, Types A | Fluarix | GlaxoSmithKline Biologicals |
| and B | | |
| Influenza Vims Vaccine, Trivalent, Types A | Fluvirin | Novartis Vaccines and Diagnostics |
| and B | | Ltd |
| Influenza Vims Vaccine, Trivalent, Types A | Agriflu | Novartis Vaccines and Diagnostics |
| and B | | S.r.l. |
| Influenza Vims Vaccine, Trivalent, Types A | Fluzone and Fluzone | Sanofi Pasteur, Inc |
| and B | High Dose | |
| Japanese Encephalitis Virus Vaccine, | Ixiaro | Intercell Biomedical |
| Inactivated, Adsorbed | | |
| Japanese Encephalitis Virus Vaccine, | JE- Vax | Research Foundation for Microbial |
| Inactivated, Adsorbed | | Diseases of Osaka University |
| Measles Virus Vaccine, Live | Attenuvax | Merck & Co. Inc |
| Measles, Mumps, and Rubella Virus Vaccine, | M-M-Vax | Merck & Co. Inc (not available) |
| Live | | |
| Measles, Mumps, Rubella and Varicella Virus | ProQuad | Merck & Co. Inc |
| Vaccine, Live | | |
| Meningococcal (Groups A, C, Y and W-13) | Menveo | Novartis Vaccines and Diagnostics, |
| Oligosaccharide Diphtheria CRM197 | | Inc. |
| Conjugate Vaccine | | |
| Meningococcal polysaccharide (Serogroups A, | Menactra | Sanofi Pasteur, Inc |
| C, Y and W-135) Diphtheria Toxoid | | |
| Conjugate Vaccine | | |
| Meningococcal polysaccharide Vaccine, | Menomune-A/C/Y/W- | Sanofi Pasteur, Inc |
| Groups A, C, Y and W-135 Combined | 135 | |
| Mumps Virus Vaccine | Mumpsvax | Merck & Co. Inc |
| Plague Vaccine | No trade name | Greer Laboratories Inc. |
| | | (not available) |
| Pneumococcal Vaccine, | Pneumovax 23 | Merck & Co, Inc |
| Pneumococcal 7-valent conjugate vaccine | Prevnar | Wyeth Pharmaceuticals |
| | | Inc |
| Diphtheria CRM197 | Prevnar 13 | Wyeth Pharmaceuticals |
| | | Inc |
| Pneumococcal 13-valent conjugate vaccine | Poliovax | Sanofi Pasteur, Ltd (not available) |
| Diphtheria CRM 197 | IPOL | Sanofi Pasteur, SA |
| Poliovirus Vaccine Inactivated (Human | Imovax | Sanofi Pasteur, SA |
| Diploid Cell) | | |
| Poliovirus Vaccine Inactivated (Monkey | RabAvert | Novartis Vaccines and Diagnostics |
| Kidney Cell) | | |
| Rabies Vaccine | No Trade Name | BioPort Corp(not available) |
| Rabies Vaccine | ROTARIX | GlaxoSmithKline Biologicals |
| Rabies Vaccine Adsorbed | RotaTeq | Merck & Co., Inc. |
| Rotavirus Vaccine, Live, Oral | Meruvax II | Merck & Co, Inc |
| Rotavirus Vaccine, Live, Oral, Pentavalent | ACAM2000 | Sanofi Pasteur Biologics Co. |
| Rubella Virus Vaccine Live | No Trade Name | MassBiologics |
| Smallpox (Vaccinia) Vaccine, Live | DECAVAC | Sanofi Pasteur, Inc |
| Tetanus & Diphtheria Toxoids Adsorbed for | TENIVAC | Sanofi Pasteur, Ltd(not available) |
| Adult Use | | |
| Tetanus & Diphtheria Toxoids Adsorbed for | No Trade name | Sanofi Pasteur, Inc |
| Adult Use | | |
| Tetanus & Diphtheria Toxoids Adsorbed for | No Trade name | Sanofi Pasteur, Inc |
| Adult Use | | |
| Tetanus Toxoid | Adacel | Sanofi Pasteur, Ltd |
| Tetanus Toxoid Adsorbed | Boostrix | GlaxoSmithKline Biologicals |
| Tetanus Toxoid, Reduced Diphtheria Toxoid | Vivotif | Berna Biotech, Ltd |
| and Acellular Pertussis Vaccine, Adsorbed | | |
| Tetanus Toxoid, Reduced Diphtheria Toxoid | TYPHIM Vi | Sanofi Pasteur, SA |
| and Acellular Pertussis Vaccine, Adsorbed | | |
| Typhoid Vaccine Live Oral Ty21a | Varivax | Merck & Co, Inc |
| Typhoid Vi Polysaccharide Vaccine | YF-Vax | Sanofi Pasteur, Inc |
| Varicella Virus Vaccine Live | Zostavax | Merck & Co., Inc. |

Additional commercially available vaccines suitable for use in the vaccines and methods of the invention may be found at, for example, www.fda.gov/BiologicsBloodVaccines/default.htm.

In some embodiments, the tubulin binding agent functions as a booster of innate or humoral immunity. In some embodiments, plinabulin functions as a booster of innate or humoral immunity.

In some embodiments, the composition described herein includes a pharmaceutically acceptable excipient.

In some embodiments, the composition is administered parenterally. In some embodiments, the composition is administered subcutaneously, intramuscularly, intravenously, or intranasaly.

In some embodiments, the composition is in a liquid or solid form.

In some embodiments, wherein the subject is a human. In some embodiments, wherein the subject is an animal. In some embodiments, wherein the subject is a mammal.

### Tubulin Binding Agent (TBA)

In some embodiments, the tubulin binding agent is selected from the group consisting of vinca alkaloids (such as vinblastine (VBL), vinorelbine (VRL), vincristine (VCR), and vindesine (VDS)), cryptophycins, dolastatins, taxanes (such as docetaxel, cabazitaxel, and paclitaxel), epothilones, discodermolides, cyclostreptin, laulimalides, taccalonolide, peloruside, hemiasterlin, combretastatins (such as combretastatin A-4 (CA-4)), colchicine, and 2-methoxyestradiol (2-ME), and pharmaceutically usable derivatives, salts, solvates, tautomers, or stereoisomers thereof, and any combinations thereof. In some embodiments, the tubulin binding agent is plinabulin. In some embodiments, the tubulin binding agent is selected from the group consisting of plinabulin, colchicine, combretastatin A-4, docetaxel, paclitaxel, vinblastine, and vincristine.

In some embodiments, the amount of the tubulin binding agent is effective to stimulate or enhance immune responsiveness in the subject to the vaccine. In some embodiments, the amount of plinabulin is effective to stimulate or enhance immune responsiveness in the subject to the vaccine.

The vaccine and the tubulin binding agent (e.g., plinabulin) described above can be formulated into pharmaceutical compositions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a vaccine described herein; (b) a safe and therapeutically effective amount of the tubulin binding agent (e.g., plinabulin); and (c) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, dextrose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

### Administration

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

Other excipient components, which may be included in the ophthalmic preparations, are chelating agents. A useful chelating agent is edetate disodium, although other chelating agents may also be used in place or in conjunction with it.

For intravenous administration, the compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

In some embodiments, the plinabulin is administered at a dose in the range of about 0.01-50 mg/m² of the body surface area. In some embodiments, the plinabulin is administered at a dose in the range of about 0.01-0.1, 0.01-0.2, 0.01-0.3, 0.01-0.4, 0.01-0.5, 0.01-0.6, 0.01-0.7, 0.01-0.8, 0.01-0.9, 0.01-1, 0.01-2, 0.01-3, 0.01-4, 0.01-5, 0.01-6, 0.01-7, 0.01-8, 0.01-9, 0.01-10, 0.01-11, 0.01-12, 0.01-13, 0.01-13.75, 0.01-14, 0.01-15, 0.01-16, 0.01-17, 0.01-18, 0.01-19, 0.01-20, 0.01-22.5, 0.01-25, 0.01-27.5, 0.01-30, 0.1-0.5, 0.1-0.6, 0.1-0.7, 0.1-0.8, 0.1-0.9, 0.1-1, 0.1-2, 0.1-3, 0.1-4, 0.1-5, 0.1-6, 0.1-7, 0.1-8, 0.1-9, 0.1-10, 0.1-11, 0.1-12, 0.1-13, 0.1-13.75, 0.1-14, 0.1-15, 0.1-16, 0.1-17, 0.1-18, 0.1-19, 0.1-20, 0.1-22.5, 0.1-25, 0.1-27.5, 0.1-30, 0.1-40, 0.1-50, 0.25-0.5, 0.25-0.6, 0. 25-0.7, 0.25-0.8, 0.25-0.9, 0.25-1, 0.25-2, 0.25-3, 0.25-4, 0.25-5, 0.25-6, 0.25-7, 0.25-8, 0.25-9, 0.25-10, 0.25-11, 0.25-12, 0.25-13, 0.25-13.75, 0.25-14, 0.25-15, 0.25-16, 0.25-17, 0.25-18, 0.25-19, 0.25-20, 0.25-22.5, 0.25-25, 0.25-27.5, 0.25-30, 0.25-40, 0.25-50, 0.5-1, 0.5-2, 0.5-3, 0.5-4, 0.5-5, 0.5-6, 0.5-7, 0.5-8, 0.5-9, 0.5-10, 0.5-11, 0.5-12, 0.5-13, 0.5-13.75, 0.5-14, 0.5-15, 0.5-16, 0.5-17, 0.5-18, 0.5-19, 0.5-20, 0.5-22.5, 0.5-25, 0.5-27.5, 0.5-30, 0.5-40, 0.5-50, 1.5-2, 1.5-3, 1.5-4, 1.5-5, 1.5-6, 1.5-7, 1.5-8, 1.5-9, 1.5-10, 1.5-11, 1.5-12, 1.5-13, 1.5-13.75, 1.5-14, 1.5-15, 1.5-16, 1.5-17, 1.5-18, 1.5-19, 1.5-20, 1.5-22.5, 1.5-25, 1.5-27.5, 1.5-30, 1.5-40, 1.5-40, 1-2, 1-3, 1-4, 1-5, 1-6, 1-7, 1-8, 1-9, 1-10, 1-11, 1-12, 1-13, 1-13.75, 1-14, 1-15, 1-16, 1-17, 1-18, 1-19, 1-20, 1-22.5, 1-25, 1-27.5, 1-30, 1-40, 1-50, 2.5-2, 2.5-3, 2.5-4, 2.5-5, 2.5-6, 2.5-7, 2.5-8, 2.5-9, 2.5-10, 2.5-11, 2.5-12, 2.5-13, 2.5-13.75, 2.5-14, 2.5-15, 2.5-16, 2.5-17, 2.5-18, 2.5-19, 2.5-20, 2.5-22.5, 2.5-25, 2.5-27.5, 2.5-30, 2.5-7.5, 3-4, 3-5, 3-6, 3-7, 3-8, 3-9, 3-10, 3-11, 3-12, 3-13, 3-13.75, 3-14, 3-15, 3-16, 3-17, 3-18, 3-19, 3-20, 3-22.5, 3-25, 3-27.5, 3-30, 3.5- 6.5, 3.5-13.75, 3.5-15, 2.5-17.5, 4-5, 4-6, 4-7, 4-8, 4-9, 4-10, 4-11, 4-12, 4-13, 4-13.75, 4-14, 4-15, 4-16, 4-17, 4-18, 4-19, 4-20, 4-22.5, 4-25, 4-27.5, 4-30, 5-6, 5-7, 5-8, 5-9, 5-10, 5-11, 5-12, 5-13, 5-13.75, 5-14, 5-15, 5-16, 5-17, 5-18, 5-19, 5-20, 5-22.5, 5-25, 5-27.5, 5-30, 6-7, 6-8, 6-9, 6-10, 6-11, 6-12, 6-13, 6-13.75, 6-14, 6-15, 6-16, 6-17, 6-18, 6-19, 6-20, 6-22.5, 6-25, 6-27.5, 6-30, 7-8, 7-9, 7-10, 7-11, 7-12, 7-13, 7-13.75, 7-14, 7-15, 7-16, 7-17, 7-18, 7-19, 7-20, 7-22.5, 7-25, 7-27.5, 7-30, 7.5-12.5, 7.5-13.5, 7.5-15, 8-9, 8-10, 8-11, 8-12, 8-13, 8-13.75, 8-14, 8-15, 8-16, 8-17, 8-18, 8-19, 8-20, 8-22.5, 8-25, 8-27.5, 8-30, 9-10, 9-11, 9-12, 9-13, 9-13.75, 9-14, 9-15, 9-16, 9-17, 9-18, 9-19, 9-20, 9-22.5, 9-25, 9-27.5, 9-30, 10-11, 10-12, 10-13, 10-13.75, 10-14, 10-15, 10-16, 10-17, 10-18, 10-19, 10-20, 10-22.5, 10-25, 10-27.5, 10-30, 11.5-15.5, 12.5-14.5, 7.5-22.5, 8.5-32.5, 9.5-15.5, 15.5-24.5, 5-35, 17.5-22.5, 22.5-32.5, 25-35, 25.5-24.5, 27.5-32.5, 2-20, t 2.5-22.5, or 9.5-21.5 mg/m², of the body surface area. In some embodiments, the plinabulin is administered at a dose of about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the plinabulin is administered at a dose less than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40 mg/m² of the body surface area. In some embodiments, the plinabulin is administered at a dose greater than about 0.01, 0.02, 0.03, 0.05, 0.07, 0.1, 0.25, 0.5, 0.75, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 32, 33, 34, 35, 36, 37, 38, 39, 40, 41, 42, 43, 44, 45, 46, 47, 48, 49, 50 mg/m² of the body surface area.

In some embodiments, the plinabulin dose is about 0.1 mg-10 mg, 0.1 mg-25 mg, 0.1 mg-30 mg, 0.1 mg-50 mg, 0.1 mg-75 mg, 0.1 mg-100 mg, 0.5 mg-10 mg, 0.5 mg-25 mg, 0.5 mg-30 mg, 0.5 mg-50 mg, 0.5 mg-75 mg, 0.5 mg-100 mg, 1 mg-10 mg, 1 mg-25 mg, 1 mg-30 mg, 1 mg-50 mg, 1 mg-75 mg,1 mg-100 mg, 2 mg-10 mg, 2 mg-25 mg, 2 mg-30 mg, 2 mg-50 mg, 2 mg-75 mg, 2 mg-100 mg, 3 mg-10 mg, 3 mg-25 mg, 3 mg-30 mg, 3 mg-50 mg, 3 mg-75 mg, 3 mg-100 mg, 4 mg-100 mg, 5 mg-10 mg, 5 mg-25 mg, 5 mg-30 mg, 5 mg-50 mg, 5 mg-75 mg, 5 mg-300 mg, 5 mg-200 mg, 7.5 mg-15 mg, 7.5 mg-25 mg, 7.5 mg-30 mg, 7.5 mg-50 mg, 7.5 mg-75 mg, 7.5 mg-100 mg, 7.5 mg - 200 mg, 10 mg-20 mg, 10 mg-25 mg, 10 mg -50 mg, 10 mg-75 mg, 10 mg-100 mg, 15 mg-30 mg, 15 mg-50 mg, 15 mg-100 mg, 20 mg-20 mg, 20 mg-100 mg, 30 mg-100 mg, 40 mg-100 mg, 10 mg-80 mg, 15 mg-80 mg, 20 mg-80 mg, 30 mg-80mg, 40 mg-80 mg, 10 mg-60 mg, 15 mg-60 mg, 20 mg-60 mg, 30 mg-60 mg, or about 40 mg-60 mg. In some embodiments, the plinabulin administered is about 20 mg-60 mg, 27 mg-60 mg, 20 mg-45 mg, or 27 mg-45 mg. In some embodiments, the plinabulin administered is about 1 mg-5 mg, 1 mg-7.5 mg, 2.5 mg-5 mg, 2.5 mg-7.5 mg, 5 mg-7.5 mg, 5 mg-9 mg, 5 mg-10 mg, 5 mg-12 mg, 5 mg-14 mg, 5 mg-15 mg, 5 mg-16 mg, 5 mg-18 mg, 5 mg-20 mg, 5 mg-22 mg, 5 mg-24 mg, 5 mg-26 mg, 5 mg-28 mg, 5 mg-30 mg, 5 mg-32 mg, 5 mg-34 mg, 5 mg-36 mg, 5 mg-38 mg, 5 mg-40 mg, 5 mg-42 mg, 5 mg-44 mg, 5 mg-46 mg, 5 mg-48 mg, 5 mg-50 mg, 5 mg-52 mg, 5 mg-54 mg, 5 mg-56 mg, 5 mg-58 mg, 5 mg-60 mg, 7 mg-7.7 mg, 7 mg-9 mg, 7 mg-10 mg, 7 mg-12 mg, 7 mg-14 mg, 7 mg-15 mg, 7 mg-16 mg, 7 mg-18 mg, 7 mg-20 mg, 7 mg-22 mg, 7 mg-24 mg, 7 mg-26 mg, 7 mg-28 mg, 7 mg-30 mg, 7 mg-32 mg, 7 mg-34 mg, 7 mg-36 mg, 7 mg-38 mg, 7 mg-40 mg, 7 mg-42 mg, 7 mg-44 mg, 7 mg-46 mg, 7 mg-48 mg, 7 mg-50 mg, 7 mg-52 mg, 7 mg-54 mg, 7 mg-56 mg, 7 mg-58 mg, 7 mg-60 mg, 9 mg-10 mg, 9 mg-12 mg, 9 mg-14 mg, 9 mg-15 mg, 9 mg-16 mg, 9 mg-18 mg, 9 mg-20 mg, 9 mg-22 mg, 9 mg-24 mg, 9 mg-26 mg, 9 mg-28 mg, 9 mg-30 mg, 9 mg-32 mg, 9 mg-34 mg, 9 mg-36 mg, 9 mg-38 mg, 9 mg-40 mg, 9 mg-42 mg, 9 mg-44 mg, 9 mg-46 mg, 9 mg-48 mg, 9 mg-50 mg, 9 mg-52 mg, 9 mg-54 mg, 9 mg-56 mg, 9 mg-58 mg, 9 mg-60 mg, 10 mg-12 mg, 10 mg-14 mg, 10 mg-15 mg, 10 mg-16 mg, 10 mg-18 mg, 10 mg-20 mg, 10 mg-22 mg, 10 mg-24 mg, 10 mg-26 mg, 10 mg-28 mg, 10 mg-30 mg, 10 mg-32 mg, 10 mg-34 mg, 10 mg-36 mg, 10 mg-38 mg, 10 mg-40 mg, 10 mg-42 mg, 10 mg-44 mg, 10 mg-46 mg, 10 mg-48 mg, 10 mg-50 mg, 10 mg-52 mg, 10 mg-54 mg, 10 mg-56 mg, 10 mg-58 mg, 10 mg-60 mg, 12 mg-14 mg, 12 mg-15 mg, 12 mg-16 mg, 12 mg-18 mg, 12 mg-20 mg, 12 mg-22 mg, 12 mg-24 mg, 12 mg-26 mg, 12 mg-28 mg, 12 mg-30 mg, 12 mg-32 mg, 12 mg-34 mg, 12 mg-36 mg, 12 mg-38 mg, 12 mg-40 mg, 12 mg-42 mg, 12 mg-44 mg, 12 mg-46 mg, 12 mg-48 mg, 12 mg-50 mg, 12 mg-52 mg, 12 mg-54 mg, 12 mg-56 mg, 12 mg-58 mg, 12 mg-60 mg, 15 mg-16 mg, 15 mg-18 mg, 15 mg-20 mg, 15 mg-22 mg, 15 mg-24 mg, 15 mg-26 mg, 15 mg-28 mg, 15 mg-30 mg, 15 mg-32 mg, 15 mg-34 mg, 15 mg-36 mg, 15 mg-38 mg, 15 mg-40 mg, 15 mg-42 mg, 15 mg-44 mg, 15 mg-46 mg, 15 mg-48 mg, 15 mg-50 mg, 15 mg-52 mg, 15 mg-54 mg, 15 mg-56 mg, 15 mg-58 mg, 15 mg-60 mg, 17 mg-18 mg, 17 mg-20 mg, 17 mg-22 mg, 17 mg-24 mg, 17 mg-26 mg, 17 mg-28 mg, 17 mg-30 mg, 17 mg-32 mg, 17 mg-34 mg, 17 mg-36 mg, 17 mg-38 mg, 17 mg-40 mg, 17 mg-42 mg, 17 mg-44 mg, 17 mg-46 mg, 17 mg-48 mg, 17 mg-50 mg, 17 mg-52 mg, 17 mg-54 mg, 17 mg-56 mg, 17 mg-58 mg, 17 mg-60 mg, 20 mg-22 mg, 20 mg-24 mg, 20 mg-26 mg, 20 mg-28 mg, 20 mg-30 mg, 20 mg-32 mg, 20 mg-34 mg, 20 mg-36 mg, 20 mg-38 mg, 20 mg-40 mg, 20 mg-42 mg, 20 mg-44 mg, 20 mg-46 mg, 20 mg-48 mg, 20 mg-50 mg, 20 mg-52 mg, 20 mg-54 mg, 20 mg-56 mg, 20 mg-58 mg, 20 mg-60 mg, 22 mg-24 mg, 22 mg-26 mg, 22 mg-28 mg, 22 mg-30 mg, 22 mg-32 mg, 22 mg-34 mg, 22 mg-36 mg, 22 mg-38 mg, 22 mg-40 mg, 22 mg-42 mg, 22 mg-44 mg, 22 mg-46 mg, 22 mg-48 mg, 22 mg-50 mg, 22 mg-52 mg, 22 mg-54 mg, 22 mg-56 mg, 22 mg-58 mg, 22 mg-60 mg, 25 mg-26 mg, 25 mg-28 mg, 25 mg-30 mg, 25 mg-32 mg, 25 mg-34 mg, 25 mg-36 mg, 25 mg-38 mg, 25 mg-40 mg, 25 mg-42 mg, 25 mg-44 mg, 25 mg-46 mg, 25 mg-48 mg, 25 mg-50 mg, 25 mg-52 mg, 25 mg-54 mg, 25 mg-56 mg, 25 mg-58 mg, 25 mg-60 mg, 27 mg-28 mg, 27 mg-30 mg, 27 mg-32 mg, 27 mg-34 mg, 27 mg-36 mg, 27 mg-38 mg, 27 mg-40 mg, 27 mg-42 mg, 27 mg-44 mg, 27 mg-46 mg, 27 mg-48 mg, 27 mg-50 mg, 27 mg-52 mg, 27 mg-54 mg, 27 mg-56 mg, 27 mg-58 mg, 27 mg-60 mg, 30 mg-32 mg, 30 mg-34 mg, 30 mg-36 mg, 30 mg-38 mg, 30 mg-40 mg, 30 mg-42 mg, 30 mg-44 mg, 30 mg-46 mg, 30 mg-48 mg, 30 mg-50 mg, 30 mg-52 mg, 30 mg-54 mg, 30 mg-56 mg, 30 mg-58 mg, 30 mg-60 mg, 33 mg-34 mg, 33 mg-36 mg, 33 mg-38 mg, 33 mg-40 mg, 33 mg-42 mg, 33 mg-44 mg, 33 mg-46 mg, 33 mg-48 mg, 33 mg-50 mg, 33 mg-52 mg, 33 mg-54 mg, 33 mg-56 mg, 33 mg-58 mg, 33 mg-60 mg, 36 mg-38 mg, 36 mg-40 mg, 36 mg-42 mg, 36 mg-44 mg, 36 mg-46 mg, 36 mg-48 mg, 36 mg-50 mg, 36 mg-52 mg, 36 mg-54 mg, 36 mg-56 mg, 36 mg-58 mg, 36 mg-60 mg, 40 mg-42 mg, 40 mg-44 mg, 40 mg-46 mg, 40 mg-48 mg, 40 mg-50 mg, 40 mg-52 mg, 40 mg-54 mg, 40 mg-56 mg, 40 mg-58 mg, 40 mg-60 mg, 43 mg-46 mg, 43 mg-48 mg, 43 mg-50 mg, 43 mg-52 mg, 43 mg-54 mg, 43 mg-56 mg, 43 mg-58 mg, 42 mg-60 mg, 45 mg-48 mg, 45 mg-50 mg, 45 mg-52 mg, 45 mg-54 mg, 45 mg-56 mg, 45 mg-58 mg, 45 mg-60 mg, 48 mg-50 mg, 48 mg-52 mg, 48 mg-54 mg, 48 mg-56 mg, 48 mg-58 mg, 48 mg-60 mg, 50 mg-52 mg, 50 mg-54 mg, 50 mg-56 mg, 50 mg-58 mg, 50 mg-60 mg, 52 mg-54 mg, 52 mg-56 mg, 52 mg-58 mg, or 52 mg-60 mg. In some embodiments, the plinabulin dose is greater than about 0.1 mg, 0.3 mg, 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg. In some embodiments, the plinabulin dose is about less than about 0.5 mg, 0.75 mg, 1 mg, 1.25 mg, 1.5 mg, 1.75 mg, 2 mg, 2.5 mg, 3 mg, 3.5 mg, 4 mg, 5 mg, about 10 mg, about 12.5 mg, about 13.5 mg, about 15 mg, about 17.5 mg, about 20 mg, about 22.5 mg, about 25 mg, about 27 mg, about 30 mg, about 40 mg, about 50 mg, about 60 mg, about 70 mg, about 80 mg, about 90 mg, about 100 mg, about 125 mg, about 150 mg, or about 200 mg.

Administration of the composition disclosed herein can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

### Method of Treatment

Some embodiments relate to a method of treatment, the method comprising administering to the subject a vaccine and a tubulin binding agent. Some embodiments relate to a method of treatment, the method comprising administering to the subject a vaccine and plinabulin.

In some embodiments, the vaccine is an infection disease vaccine. In some embodiments, the vaccine is a cancer vaccine.

Some embodiments relate to a method of enhancing an immune response to a vaccine in a subject, said method comprising administering to the subject a vaccine and the tubulin agent (e.g., plinabulin), wherein the immune response to the vaccine is enhanced compared to the immune response generated by administration of the vaccine alone to the subject

Some embodiments relate to a method of inducing lymphocyte cell proliferation, comprising administering an effective amount of the tubulin agent (e.g., plinabulin) and a vaccine to a subject in need thereof.

Some embodiments relate to a method of inducing B cell proliferation, comprising administering an effective amount of the tubulin agent (e.g., plinabulin) and a vaccine to a subject in need thereof.

Some embodiments relate to a method of inducing a production of Immunoglbulin, comprising administering an effective amount of the tubulin agent (e.g., plinabulin) and a vaccine to a subject in need thereof. In some embodiments, the immunoglobulin is selected from the group consisting of IgG, IgM, IgA, IgD, and IgE.

Some embodiments relate to a method of enhancing an immune response in a cancer treatment, comprising administering to the subject a cancer vaccine and the tubulin agent (e.g., plinabulin), wherein the immune response to the cancer vaccine is enhanced compared to the immune response generated by administration of the vaccine alone to the subject.

The vaccine and the tubulin agent (e.g., plinabulin) can be administered either separately (e.g., the vaccine can be administered before or after plinabulin is administered to the subject) or as a single formulation (e.g., the vaccine can be administered simultaneously with plinabulin). In some embodiments, the method described herein includes administering the tubulin agent (e.g., plinabulin) and the vaccine simultaneously. In some embodiments, the method described herein includes administering the tubulin agent (e.g., plinabulin) prior to or after administering the vaccine.

Some embodiments relate to a method of enhancing an immune response, the method comprising administering a subject with a vaccine and administering the subject with a tubulin binding agent after the vaccine administration. In some embodiments, the tubulin binding agent can be plinabulin. In some embodiments, the tubulin binding agent can be selected from the group consisting of Vinca Alkaloids, Cryptophycins, Dolastatins, Taxanes, Epothilones, Discodermolides, Cyclostreptin, Laulimalides, Taccalonolide, Peloruside, Hemiasterlin, Combretastatins, Colchicine and 2 methoxyestradiol.

In some embodiments, the tubulin binding agent (e.g., plinabulin) is administered at least 30 mins, 1 h, 2 h, 3 h, 4 h, 5 h, 6 h, 7 h, 8 h, 9 h, 10 h, 11 h, 12 h, 15 h, 18 h, 20 h, 24 h, 36 h, 2 days, 3 days, 4 days, 5 days, 6 days, 7 days, 8 days, 9 days, or 10 days after the administration of vaccine. In some embodiments, the tubulin agent (e.g., plinabulin) is administered no later than 1 day, 2 days, 3 days, 4 days, 5 days, 6 days, 7 days, 8 days, 9 days, 10 days, 12 days, 15 days, or 20 days after the administration of vaccine. In some embodiments, the tubulin agent (e.g., plinabulin) is administered 1 h-1 day, 1 h-2 days, 1 h-3 days, 1 h-4 days, 1 h-5 days, 1 h-6 days, 1 h-7 days, 1 h-8 days, 1 h-9 days, 1 h-10 days, 1 day-2days, 1 day-3 days, 1 day-4 days, 1 day-5 days, 1 day-6 days, 1 day-7 days, 1 day-8 days, 1 day-9 days, 1 day-10 days, 2 days-3 days, 2 days-4 days, 2 days-5 days, 2 days-6 days, 2 days-7 days, 2 days-8 days, 2 days-9 days, 2 days-10 days, 3 days-4 days, 3 days-5 days, 3 days-6 days, 3 days-7 days, 3 days-8 days, 3 days-9 days, 3 days-10 days, 4 days-5 days, 4 days-6 days, 4 days-7 days, 4 days-8 days, 4 days-9 days, 4 days-10 days, 5 days-6 days, 5 days-8 days, 5 days-10 days after the administration of vaccine.

Some embodiments relate to a method of preparing the composition described herein, comprising combining the tubulin agent (e.g., plinabulin) and the vaccine.

### EXAMPLES

### Example 1

### Plinabulin Enhances B-Cell Activation in BCG Therapy

A study is performed to evaluate use of Plinabulin during the induction phase of intravesical BCG (Bacillus Calmette-Guérin) therapy for high grade non-muscle invasive urothelial carcinoma of the bladder to allow for increasing the dose of BCG that is installed in the bladder. Plinabulin promotes presentation of the BCG antigen to immune-competent cells, thus therefore enhancing the immunotherapeutic potential of BCG. It was shown that administration of BCG induces anti-BCG-antibody production (IgM and IgG) by activated B-lymphocytes. With the administration of Plinabulin, an increased BCG-specific B-Lymphocyte activation occurs. The addition of Plinabulin may lead to improved BCG efficacy, and if needed, allowing BCG dose escalation whilst promoting better treatment response.

A Phase I/II Study of Plinabulin with Double Dose Bacillus Calmette-Guerin (BCG) Induction Therapy for High Grade Non-Muscle Invasive Urothelial Carcinoma of the Bladder: a Study of Safety and Non-Inferiority is performed.

The primary objective of Phase I study is to determine the maximum tolerated dose of plinabulin in combination with double dose BCG in patients with high grade transitional cell carcinoma of the bladder. The primary objective of phase II study is to prove non-inferiority of maximum tolerated dose of plinabulin in combination with double dose BCG by achieving at least 50% response rate at 3 months (no visualization of tumor and negative cytology).

Some secondary Objectives include assessing the efficacy of the treatment at a moderate follow-up time interval (RFS and PFS at 1 year), and assessing changes in subject quality of life, bladder irritation and pain, as well as overall health and wellness during the treatment and follow up to 1 year.

Some Correlative/Exploratory Objectives include determining the time course and magnitude and time of onset of anti-BCG IgM and IgG levels over the treatment period with BCG instillation and plinabulin intravenous infusion, and exploring urinary cytokine production (INF-g, IL-1, IL-2, IL-6, IL-10, IL-12p70, TNF-a) upon the treatment of BCG instillation and plinabulin intravenous infusion.

This is an open-label Phase I/II study, with a dose escalation part (Phase I) and a 1-arm efficacy study (Phase II) in patients with High Grade Non-Muscle Invasive Urothelial Carcinoma of the Bladder.

Phase I: A maximum tolerated Plinabulin dose (MTD) determination. Eligible patients receive plinabulin intravenously at one of four escalating dose cohorts from 5 mg/m² to 30 mg/m² in combination with double dose BCG instillation. This phase determines the toxicity and MTD of adjuvant therapy of Plinabulin and BCG. At least 3 patients are enrolled in each cohort, starting at 5 mg/m² of plinabulin with double dose BCG. The dose of plinabulin is escalated in sequential patient cohorts after the safety data from first 21 days after initial study drug administration is reviewed. The MTD dose is determined as the highest dose cohort with either zero out of three patients or less than two out of six patients experiencing any Dose Limiting Toxicity (DLT). This MTD will be the dose for the phase II trial, also called Recommended Phase 2 Dose (RP2D).

Phase II: Efficacy study. The Plinabulin dose selected from the above phase I is passed to phase II. Patients from phase I, receiving the MTD dose, are incorporated into the second phase. The patients are assessed for recurrence of tumor (primary efficacy endpoint), defined as evidence of tumor on office cystoscopy and positive urine cytology at 3 months, following 6-weeks of induction double dose BCG and Plinabulin therapy. For efficacy assessment, there are two evaluations and if there are 11/18 or less, or 26/36 or less, patients demonstrating response, the trial will be stopped, and the treatment considered not active enough. The minimum response rate goal for the study is 50% response rate (no visualization of tumor and negative urine cytology post-induction therapy) at 3 months and desired response rate is 70% at 3 months. Response rate below 50% is considered unacceptable. The total number of individuals required for both phase I/II study is 54 individuals or less depending on the toxicity and the response assessment.

For Phase I, the primary endpoints for phase I are the incidence and severity of AEs/SAEs and treatment discontinuations due to AEs. For Phase II, the primary end points is RFS at 3 months post-induction therapy (recurrence free survival; i.e. non-visualization of tumor and negative urine cytology).

The secondary endpoints include: Phase II: RFS at 1 year (recurrence free survival; i.e. non-visualization of tumor and negative urine cytology); Phase II: PFS at 1 year (progression free survival; i.e. non-upstaging of tumor on future TURs); Phase I and II: Change in Quality of Life [Time Frame: change from baseline to 6 weeks, and 3, and 12 months after starting treatment] measured using the American Urologic Association Symptoms Index (AUA IPSS); Phase I and II: Change in Quality of Life [Time Frame: change from baseline to 6 weeks, and 3, and 12 months after starting treatment] using the Quality of Life (QOL) questionnaire; Phase I and II: Change in bladder irritation and pain [Time Frame: change from baseline to 6 weeks, and 3, and 12 months after starting treatment] using O'Leary-Sant Indices; Phase I and II: Change in overall health and wellness [Time Frame: change from baseline to 6 weeks, and 3, and 12 months after starting treatment] using Functional Assessment of Cancer Therapy-Bladder (FACT-BL).

The correlative/Exploratory endpoints include: in phase I and II: Anti-BCG IgM and IgG levels (titers) over time; and in phase I and II: Urinary cytokine (INF-g, IL-1, IL-2, IL-6, IL-10, IL-12p70, TNF-a) levels over time.

Phase I involves a maximum tolerated Plinabulin dose (MTD) determination study. This phase is a dose escalation study to determine the toxicity and MTD of adjuvant therapy of Plinabulin and BCG.

Phase II involves establishing efficacy study. The Plinabulin dose selected from the above phase I is passed to phase II. Patients from phase I, receiving the optimal dose, are incorporated into the second phase. The patients are assessed for recurrence of tumor (primary efficacy endpoint), defined as evidence of tumor on office cystoscopy and positive urine cytology at 3 months, following 6-weeks of induction double dose BCG and Plinabulin therapy.

Phase I Dosing Regimen: The dosing regimen follows the standard regimen for double dose BCG induction therapy, i.e. following a rest-period of 2 weeks after the initial TUR (during which time the pathology results also become available), the eligible patients start a 6-week course of once-weekly double dose BCG with Plinabulin. The patients then return at 3 months from their first intravesical BCG treatment (or 6 weeks from the last double dose BCG+Plinabulin treatment) for evaluation of tumor recurrence (via cystoscopy and urine cytology).

TABLE 1
Dose Escalation Schedule
Table 1. Study Cohorts for MTD determination

| Cohort No. | # Subjects | Plinabulin Dose | | BCG dose |
|---|---|---|---|---|
| 1 | 3 to 6 | 5 | mg/m² | Double |
| 2 | 3 to 6 | 13.5 | mg/m² | Double |
| 3 | 3 to 6 | 20 | mg/m² | Double |
| 4 | 3 to 6 | 30 | mg/m² | Double |

TABLE 2
Dose Escalation Decision Rules

No. of Patients

| No. of Toxicities | 3 | 4 | 5 | 6 |
|---|---|---|---|---|
| 0 | E | E | E | E |
| 1 | S | S | S | E |
| 2 | D | S | S | S |
| 3 | U | U | D | S |
| 4 | NA | U | U | U |
| 5 | NA | NA | U | U |

E=Escalate to next dose level, S=Stay in the current dose level, D=De-escalate to 1 lower dose level, U=De-escalate to 1 lower dose level without returning to current dose level, At the end of the planned enrollment to the Phase 1 part of the study, a complete review of the safety data will occur. The recommended Phase 2 dose (RP2D) will be determined, and the Phase 2 part initiated.

Phase I Dose Escalation: The phase I can have 3 cohorts of 3 to 6 subjects, with the dosing starting in cohort 1 at a dose of 5 mg/m² Plinabulin+double dose BCG. Three more subjects are added if 1 of 3 patients experience dose limiting toxicity (side effects Grade>2). The dose is escalated in the next cohort (cohort 2) if 0 of 3 or <2/6 of patients experienced the dose limiting toxicity in cohort 1 (Grade>2). The MTD dose is determined as the highest dose cohort with either 0/3 or <2/6 toxicity. Only one dose de-escalation of BCG is allowed at the lowest dose of Plinabulin (5 mg/m2). For example: If toxicity blocked advancement of combined 13.5 mg/m² of Plinabulin with double dose BCG to next level of phase I then the combination of 5 mg/m² of Plinabulin with double dose BCG will be advanced to phase II of this study instead of continuing to examine 13.5 mg/m² of Plinabulin with single dose BCG in another level of the phase I part of this trial. A maximum of 24 patients are needed to complete the phase I portion of the study. An optimal dose (either MTD or lower) is used for the phase II trial, and the patients who received the optimal dose are included as part of phase II of the trial.

Dose-limiting toxicities (DLTs) are assessed for each patient during the 21 days following their first Plinabulin and double dose BCG dose. Any suspected or confirmed DLT should be reported immediately (within 24 hours) to the Principal Investigator. A DLT is defined as the following treatment related AEs or laboratory abnormalities, graded according to NCI CTCAE version 5.0: Grade 4 anemia unrelated to underlying disease; Grade 3 thrombocytopenia with clinically significant bleeding or grade 4 thrombocytopenia lasting more than 7 days and/or requiring a platelet transfusion; Grade 4 neutropenia lasting more than 7 days; ≥Grade 3 non-hematologic AEs, except for the exclusions listed below; ≥Grade 3 nausea, vomiting, diarrhea, or electrolyte imbalances lasting>48 hours despite optimal prophylactic and curative treatment; ≥Grade 3 hypersensitivity reaction (unless first occurrence and resolves within 6 hours with appropriate clinical management); Treatment delay>21 days secondary to recovery from study drugs-related AEs.

Drug Administration: The calculated dose (mg) of Plinabulin (at a concentration of 4 mg/mL in the vial) is diluted in dextrose 5% in water (D5W) and administered IV with an in-line filter peripherally or centrally. The diluted dose is used within 4 hours of dilution. Plinabulin is protected from light at all times (storage, prior to, during and after dilution). Syringe: PVC-free, light protective, amber syringe, greater than 10 mL is suggested, for transfer of plinabulin drug product into D5W bag. If PVC-free, light protective, amber syringes are not available, please ensure that exposure to light is at a minimum. Transfer time from the Plinabulin vial to the amber sleeve covered D5W bag should be kept to the minimum, and not exceed 1 minute. Instructions for pharmacy drug preparation can be found in the study Pharmacy Manual. The Plinabulin dose should be calculated based on the baseline BSA. If BSA subsequently varies from baseline by more than ±10%, then the newer BSA value should be used for calculation of subsequent doses. Dose of Plinabulin can be in a range of doses, such as 0.1 mg/m² to 100 mg/m².

BCG Treatment: BCG treatment is according to Institutional standard for the site; BCG would be delivered via a urethral catheter.

Dose Selection: The rationale for the use of double dose of BCG is based upon multiple studies that have shown that BCG therapy has higher efficacy when used at higher doses and/or for longer periods in patients with NMIBC. However, higher dosing is limited by concomitant toxicities, because with increasing dose, the toxicities of BCG also increase. The use of Plinabulin, the BCG treatment-related inflammation and the associated side effects can be mitigated and therefore allow higher dosing of BCG.

Two vials of BCG suspended in 50 ml preservative-free saline will our dose for this study. The preparation of the BCG suspension is completed using aseptic technique and according to FDA approved labeling and use information.

Patients are asked to report any fever or flu-like symptoms to their treating Investigator immediately, as well as any systemic manifestations increasing in intensity with repeated instillations, or local symptoms (frequency, urgency, burning sensation with urination) lasting more than 2-3 days. If a patient develops persistent fever or experiences an acute febrile illness consistent with BCG infection, BCG treatment can be discontinued and the patient immediately evaluated and treated for systemic infection.

Monitoring for Systemic Dissemination of BCG: To ensure early identification of systemic BCG infection, subjects will be monitored for symptoms of systemic infection. For each cycle of BCG treatment, subjects will be telephoned on Days 2-4 (Treatment is Day 1) of Treatment Weeks 1-6 to inquire about any symptoms they may be experiencing. In addition, each patient will be provided with a thermometer and diary and asked to record oral temperatures each morning and evening throughout the BCG treatment (Weeks 1-6) and for recording of any other symptoms they may be experiencing.

Timing of Dose Administration: BCG treatment should be administered beginning on day 1 of the 6-week cycle. BCG treatment is repeated every 7 days at weeks 2, 3, 4, 5, and 6. BCG treatment may be administered up to 1 day before or after the scheduled date (at 7 days) due to administrative reasons. All trial treatments are administered on an outpatient basis and according to institutional standards.

TREATMENT PLAN: Study treatment is administered as a 6-week cycle with once per week of intravesical double dose BCG and intravenous Plinabulin. For patients with a body surface area (BSA) greater than 2.4 m², dosing should be calculated using a maximum BSA of 2.4 m² for Plinabulin. BCG dosing is as follows: 2 vials of TICE strain, each containing 5×10̂⁸ CFU, will be suspended in 50 cc of normal saline, thus, achieving double dose strength (the usual full-dose of BCG is 1 vial of TICE strain containing 5×10̂⁸ CFU suspended in 50 cc of normal saline).

### Example 2

### Plinabulin Enhances B-Cell Response to Ovalbumin Immunization

A study was performed to evaluate the effect of plinabulin on boosting B-cell response to immunization. Samples were prepared using an emulsion of complete Freund's adjuvant (CFA) with the foreign protein ovalbumin (OVA), each with varying concentrations of plinabulin (plinabulin doses range from 0.01 mg to 30 mg), and the control was prepared with no plinabulin added. Normal healthy mice were immunized by subcutaneous injection of the CFA+OVA emulsion +/−plinabulin, and/or intraperitoneal injection of ALUM+OVA adjuvant +/−plinabulin (n=5 mice per plinabulin dose group). At different time points as discussed below in this Example, the animals were bled to collect serum for evaluating the concentration of IgG that binds OVA in the serum. Mice immunized with plinabulin showed higher concentrations of anti-OVA IgG than mice immunized with CFA+OVA or ALUM+OVA (OVA emulsified in alum adjuvant) without plinabulin, indicating that plinabulin can boost B-cell responses to immunization.

DOSE PREPARATION OF OVALBUMIN EMULSIONS IN COMPLETE FREUND'S ADJUVANT: EndoFit™ Ovalbumin Kit (InvivoGen, USA), containing 10 mg powder in one glass vial and 10 mL of sterile endotoxin-free physiological water in one glass vial, and CFA 10 mL in one glass vial were received in good condition and stored at 2-8° C. until Day 1. The sterile endotoxin-free saline solution was allowed to reach room temperature before use. Inside a BSL2 cabinet, 5 mL sterile water was added into the vial containing 10 mg of OVA and gently agitated to obtain a homogenous 2 mg/mL OVA solution. To 2.5 mL of CFA, 2.5 mL of OVA solution was added; and the mixture was emulsified by vigorously mixing into two connected 10 mL locking syringes to prepare a 1 mg/mL OVA/CFA emulsion. The emulsification was kept chilled by placing the apparatus into crushed ice for 5 minutes, then mixing and cooling was repeated an additional two times. Stability of the emulsion was tested by adding a drop into water to verify that the emulsion would not dissipate. The emulsion was transferred into five 1 mL syringes for injection and kept cool on crushed ice until immunization. The emulsification processes were repeated with an additional 2.5 mL of OVA solution and 2.5 mL of CFA during immunization of animals to use within 4 hours of preparation.

TABLE 3
Formulations Exemplified in the Study of Example 2

| | Vaccine | Test Substance | | |
|---|---|---|---|---|
| Formulations | OVA in CFA | Plin-A | Plin-B | Vehicle Control |
| Composition/ | OVA-CFA | Plinabulin: | Plinabulin: | 7.1% Tween-80^{M}, |
| Concentration | Emulsification: | 0.75 mg/mL | 1.0 mg/mL | 25.5% propylene |
| | 1 mg/mL | | | glycol, 67.4% D5W |
| | | | | (5% dextrose in |
| | | | | water), all volume |
| | | | | percentages |
| Storage | OVA: −20° C.; | 15-25° C. | 15-25° C. | 15-25° C. |
| Conditions | CFA: 4° C.; | (protected | (protected | |
| | Emulsion used | from light) | from light) | |
| | within 4 hours | | | |
| | of preparation | | | |
| Dose | 100 μL/mouse | 10 mL/kg | 10 mL/kg* | 10 mL/kg |
| Route | SC | IP | IP | IP |

*Dosing of plinabulin was reduced to 10 mL/kg following the death of three mice after initial dosing of 15 mL/kg.

DOSE PREPARATION OF VEHICLE CONTROL: The vehicle (control) was prepared on each day of dosing by adding 284 μL of Tween-80 (polyoxyethylene sorbitan monooleate; Sigma, USA) to an amber vial using a micropipette and was vortexed for 1 min. 1,020 μL of propylene glycol was then added to the amber vial and vortexed for 15 min, followed by 30-min of sonication in a water bath. Finally, 2,696 μL of 5% dextrose in water (D5W) was added to the solution and was vortexed for 3 min to achieve a 4 mL of vehicle solution composed of 7.1% Tween 80 (v/v), 25.5% propylene glycol (v/v), and 67.4% D5W (v/v).

DOSE PREPARATION OF TEST ARTICLES: Plinabulin solution (Plin-A) at a 0.75 mg/mL was prepared on each day of dosing. Plinabulin powder (3 mg) was weighed into a separate light protected amber vial at room temperature. Using a micropipette, 284 μL of Tween-80 was added to the amber vial and vortex for 1 minute. 1,020 μL of propylene glycol was added to the amber vial and vortexed for 15 minutes, then sonicated in a water bath for 30 minutes. Finally, 2,696 μL of 5% dextrose in water was added to the solution and vortexed for 3 minutes to achieve 4 mL of a 0.75 mg/mL plinabulin solution. Plinabulin solution (Plin-B) at a 1.0 mg/mL was prepared on each day of dosing. Plinabulin powder (3 mg) was weighed into a separate light protected amber vial at room temperature. Using a micropipette, 213 μL of Tween-80 was added to the amber vial and vortex for 1 minute. 765 μL of propylene glycol was added to the amber vial and vortex for 15 minutes, then sonicated in a water bath for 30 minutes. Finally, 2,022 μL of 5% dextrose in water was added to the solution and vortexed for 3 minutes to achieve 3 mL of a 1.0 mg/mL plinabulin solution.

The formulations in the study of Example 2 are summarized in Table 3 below herein. Unused formulated test article (solutions) were stored at −80° C. for potential analysis of plinabulin concentration.

ANTI-OVALBUMIN ASSAY: Mouse anti-OVA IgG₁ ELISA kits (Cayman Chemical, USA) were purchased to perform the assay. Serum samples from Day 30 were assayed in 1-to-2,000, 1-to-6,000 and 1-to-20,000 dilution. Serum samples from Day 62 were assayed in 1-to-2,000 and 1-to-20,000 dilution.

ASSAY REAGENTS AND STANDARD PREPARATION: All reagents were brought to room temperature before used. Assay buffer was prepared by diluting the content of one vial of immunoassay Buffer B concentrate (10×) with 90 mL of water. The vial was rinsed to remove any salts that may have precipitated. Wash buffer was prepared by adding 5 mL of Wash Buffer concentrate and 1 mL of Polysorbate 20 to deionized water to prepare 2,000 mL of wash buffer. The Standard was prepared by reconstitution with 1 mL of assay buffer to make a stock solution of 200 ng/mL and was gently mixed for 15 minutes. For the standard curve, the stock 200 ng/mL was the highest concentration, and the assay buffer was the zero standard: 0 ng/ml. To do the serial dilution, eight tubes were labeled No. 1 through No. 8 and 250 μL of the assay buffer was added to Tubes Nos. 2-8. 500 μL of stock solution (200 ng/mL) was added to Tube No. 1. A pipette was used to transfer 250 μL of solution from Tube No. 1 and added to Tube No. 2 and mixed gently. Next, 250 μL of solution was removed from Tube No. 2 and added to Tube No. 3. Tube No. 3 was mixed gently. This process was repeated for Tubes Nos. 4-8.

SERUM SAMPLES PREPARATION: Serum samples were removed from −80° C. freezer and thawed on wet ice for 1 hr. Serum samples from Day 30 were assayed in 1:2,000, 1:6,000 and 1:20,000 dilutions; and serum samples from Day 62 were assayed in 1:2,000 and 1:20,000 dilutions with assay buffer.

REPRESENTATIVE ASSAY PROCEDURE: 100 μL of samples, standard, control, or diluted, were added to the respective sample wells according to pre-determined ELISA plate maps. The plates were covered with the adhesive strips and incubated for 2 hours at room temperature on a horizontal orbital microplate shaker. After 2-hrs incubation, wells were washed four times with 400 μL of wash buffer. Following the last wash, the remaining wash buffer was removed via decantation. The plates were then inverted and blotted against clean paper towels. 100 μL of Goat anti-mouse IgG1 HRP conjugate were added to each well. The plates were covered with a new adhesive strip and incubated for another 1 hr at room temperature on a horizontal orbital microplate shaker. After 1-hr incubation, wells were washed four times again with 400 μL of wash buffer and then 100 μL of TMB substrate solution was added to each well. The plate was incubated for 30 minutes at room temperature while protected from light. After 30 minutes incubation, 100 μL of stop solution was added to each well. The optical density of each well was determined within 30 minutes using a microplate reader (SpectraMax i3X, Molecular Devices) set to 450 nm.

ANIMAL TESTING SET-UPS:

Species and Strain: Mouse, C57BL6.

Sex and Number: Female, 25/group, 3 groups, 5 replacements (80 total mice).

Ages: Mice were approximately 5 weeks old on arrival. Mice were born Sept. 4, 2018 (±3 days).

Body Weights: Mice body weight ranged from 15.30 to 19.70 g on Day 1.

Source: Mice were purchased from Jackson Laboratory.

Identification: Individual mice were identified ear tag. Cage cards were affixed to each cage designating the IACUC protocol number, vendor, species/strain, sex, group designation, and individual animal study numbers.

Dropouts and Replacements: There were no dropouts or replacements used on this study prior to immunization.

Justification for Test System and Number of Animals: C57BL6 mice were selected for this study based on their historical use as low-order animals for serial blood collections. The number of animals requested was based on scientific rationale, regulatory requirements and statistical consideration. The number of animals used for this study was the minimum required to produce interpretable data for decision making. Regulatory agencies indicated that, in general, a group size of 3 to 15 animals/group was sufficient to detect test article related effects in a well-designed study. Sponsor had determined appropriate group size for assessment of their test articles; 5 animals/group, with 3 groups in each of 5 study subgroups.

All animal housing and research procedures involving live animals was performed at animal research facilities accredited by the Association for Assessment and Accreditation of Laboratory Animal Care International (AAALAC) International. The standards for animal husbandry and care were those found in the U.S. Department of Agriculture's (USDA) Animal Welfare Act (9 CFR Parts 1, 2, and 3), The Guide for the Care and Use of Laboratory Animals (8th Edition, Revised 2011, National Academy Press, Washington, DC, 2011) and the Standard Operating Procedures (SOPs) of the research facilities.

Housing: Throughout the study, mice were pair-housed, 2-5 per cage, in polycarbonate cages with absorbent bedding material. The cages conformed to standards set forth in The Guide for the Care and Use of Laboratory Animals.

Enrichment: Mice were provided enrichment items (nesting and housing materials) as per SOPs of the research facilities.

Acclimation: Mice were acclimated at least 3 days following arrival at the research facilities.

Veterinary Care: The Attending Veterinarian was on-call during the live animal phase of the study.

Temperature: Environmental controls were set to maintain temperatures from 18° C. to 29° C.±3° C.

Humidity: Environmental controls were monitored and as closely as possible to maintain a range of 30% to 70% humidity ±5%.

Light: The light source was lighting on a 12 hr/12 hr on/off cycle except as required for specimen collection and study conduct.

Concurrent Medication: No concurrent medication was given on this study.

Feed: Mice were provided with Envigo Teklad rodent diet 2018C (Lot #: 05212018; Expiration date: 21 Nov. 2018).

Water: Mice were provided with municipal tap water ad libitum. The water was offered via refillable water bottles. The municipal water supplying the laboratory (San Diego City Water Department, San Diego, Calif., USA) was regularly analyzed for contaminants per SOPs of the research facilities to ascertain that none were present at levels that would negatively impact the results of the study.

Contamination Statement: No known contaminants in the feed, water, or bedding were expected to interfere with the test article in this study.

Sanitation: Room and equipment sanitation procedures were conducted in accordance with applicable SOPs of the research facilities and with guidelines as stated in The Guide for the Care and Use of Laboratory Animals. Staff wore respirator and appropriate personal protective equipment.

Handling of Clinically Ill, Moribund, or Found-Dead Animals: The decision to euthanize clinically ill or moribund mice was the responsibility of the Study Director, in possible collaboration with the Attending Veterinarian and the Sponsor's Study Monitor or their designees where possible. Methods for euthanasia were used in accordance with American Veterinary Medical Association Guidelines for The Euthanasia of Animals: 2013 Edition (J. Am. Vet. Med. Assoc., 218:669-696, 2013). Mice found dead or moribund were grossly necropsied at the request of the Study Director and disposed of per SOPs of the research facilities.

Euthanasia: Mice were anesthetized via inhaled isoflurane prior to tissue collection. Mice were exposed to 2-5% isofluorane until deep anesthesia occurred, as confirmed using reflexive pinching techniques. As part of sample collection design, mice were exsanguinated using a 25G needle and syringe inserted into the heart without dissection. This blood collection and cervical dislocation served as the secondary method of confirming death prior to carcass disposition.

DOSING SCHEDULE:

TABLE 4
Dosing Schedule Exemplified in the Study of Example 2

| | | | Dose of Plinabulin | | | | |
|---|---|---|---|---|---|---|---|
| Test | | Test | Concentration | Volume | Mass | | |
| Group | | Substance | (mg/mL) | (μL/g) | (mg/kg) | Dosing Regime^{‡} | Route |
| 1 | I | Vehicle Control | 0 | 10 | 0 | BID 3 hours apart | IP |
| 2 | | Plinabulin-A | 0.75 | 10 | 7.5 | (±10%), 1 hour after | IP |
| 3 | | Plinabulin-B | 1.0 | 15* | 15* | immunization on Day 1 | IP |
| 1 | II | Vehicle Control | 0 | 10 | 0 | BID 3 hours apart | IP |
| 2 | | Plinabulin-A | 0.75 | 10 | 7.5 | (±10%) on Day 3 | IP |
| 3 | | Plinabulin-B | 1.0 | 10 | 10 | | IP |
| 1 | III | Vehicle Control | 0 | 10 | 0 | BID 3 hours apart | IP |
| 2 | | Plinabulin-A | 0.75 | 10 | 7.5 | (±10%) on Day 6; | IP |
| 3 | | Plinabulin-B | 1.0 | 10 | 10 | | IP |
| 1 | IV | Vehicle Control | 0 | 10 | 0 | BID 3 hours apart | IP |
| 2 | | Plinabulin-A | 0.75 | 10 | 7.5 | (±10%) on Day 14 | IP |
| 3 | | Plinabulin-B | 1.0 | 10 | 10 | | IP |
| 1 | V | Vehicle Control | 0 | 10 | 0 | BID 3 hours apart | IP |
| 2 | | Plinabulin-A | 0.75 | 10 | 7.5 | (±10%) on Day 28 | IP |
| 3 | | Plinabulin-B | 1.0 | 10 | 10 | | IP |

*Dosing of plinabulin in Groups 3-II, 3-III, 3-IV, and 3-V was reduced from 15 to 10 mg/kg following the death of three mice after initial dose administration in Group 3-I.
^{‡}In each test group, a 100 μg dose of OVA in CFA emulsification was administered via subcutaneous injection at 100 μL per mouse on Day 1.

Duration of Study: The live-phase portion of this study was 62 days, not including acclimation.

Randomization: Mice were formally randomized by body weight into treatment groups on the day of immunization.

Fasting: Mice were not fasted on this study.

Immunization Administration: All 75 mice received a 100 μg dose of OVA in CFA emulsification at 100 μL per animal via subcutaneous administration on Day 1 (17 Oct. 2018) of the study. The subcutaneous dose was on the dorsal surface of the animal and administered with a 25G needle. The OVA/CFA emulsification was kept on wet ice between administrations and was used within 4 hours of each preparation.

Test Article Administration: Each animal had test material administered intraperitonially via a 26G needle at 10 or 15 μL/g per dosing as indicated in Table 4 below. The dosing date, relative to the date of immunization (17 Oct. 2018), is also indicated in Table 4.

Justification for Administration of Immunization and Test Material: Injection with OVA in CFA was administered subcutaneously (SC) to induce immunization. Test article (e.g., plinabulin) was administered via IP dosing per sponsor's request. The IP route was used to deliver the test material as test material has proven in previous studies to have good pharmacokinetics with this route. The test material was delivered twice in one day (BID), 3 hours apart, to better model the pharmacokinetics seen in patients (plasma elimination half-life in mice is ˜1.5-2 hours, versus ˜5-6 hours in human).

OBSERVATIONS, MEASUREMENTS, AND SPECIMENS:

Physical Examinations: A qualified Study Investigator conducted general physical examinations prior to dosing. The general examination included, but was not limited to assessment of skin, mobility, external orifices, behavior, and reaction to external stimuli. Physical examinations were conducted on 11 Oct. 2019. All animals were normal and deemed healthy prior to randomization on study.

Moribundity/Morbidity: All animals were observed for mortality/moribundity twice daily (morning and afternoon) during the week days, and once daily on weekends or holidays.

Detailed Clinical Observations: Scheduled detailed clinical observations were not conducted on this study. Unscheduled clinical observations were made on mice flagged by trained personnel during daily moribundity/morbidity checks for any reason.

Body Weights: Body weights were measured once weekly. On days when any animals in the study are treated, body weights were measured in all mice in the study.

Food Consumption: Food consumption was not recorded in this study.

Blood Sample Collection: Whole blood samples were collected into clot activator tubes either via submandibular vein or retro-orbital at 100 μL per collection on Day 1 (prior to immunization), Day 8, and Day 30. A maximum volume of whole blood was collected into clot activator tubes via cardiac puncture at termination on Day 62. All blood samples were allowed to clot at room temperature, centrifuged ambient (approximately 20-25° C.) at 3,000 RPM for 10-15 minutes, and serum supernatant was transferred into clean cryovials for each serum sample. Serum supernatant was stored frozen at −80° C. (±12° C.) until ready for analysis.

Measurement of IgG Antibody Anti-OVA: Mouse OVA specific antibody was measured by ELISA. IgG1 anti-OVA was measured using commercially available ELISA kit (Cat. #: 500830, Cayman Chemical quantitative ELISA) following Day 30 sample collection, and then again following Day 62 sample collection.

Euthanasia, Tissue Collection, & Early Death/Unscheduled Sacrifice: Replacement animals were euthanized within 48 hrs of the last treatment dose of the last subgroup. All surviving animals were euthanized for terminal blood collections on Day 62. Signs of illness or moribundity/morbidity were documented. The Study Director, in consultation with the Study Monitor, determined euthanasia was required for mice in extremis. Methods for euthanasia were used in accordance with AVMA Guidelines for the Euthanasia of Animals: 2013 Edition. Mice found dead or moribund were discarded at the request of the Study Director.

RESULTS:

UNSCHEDULED CLINICAL OBSERVATIONS/MORTALITY CHECKS: Three animals in subgroup I, group 3 (Animal #s: 3501, 3503, 3505) dosed at 15 mg/kg were found with clinical observations including, decreased activity, irregular breathing, dehydration, and body cold to touch within 1 day following test article administration. To the extent that the death of three animals found during the study period may be related to the plinabulin treatment, subsequent dose of plinabulin was lowered. The remaining subgroups II-V, group 3 animals were dosed at 10 mg/kg (indicated as “*” in Tables 3-4 above). The experimental observations described below in this Example were focused on the lower dose (10-mg/kg) mice.

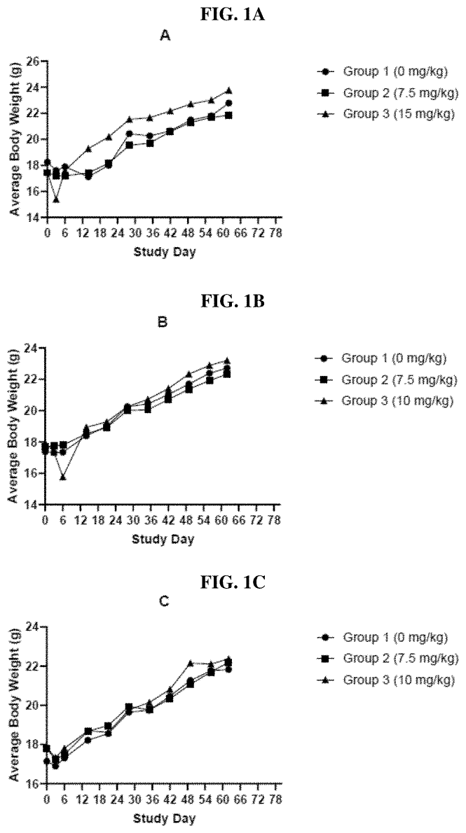

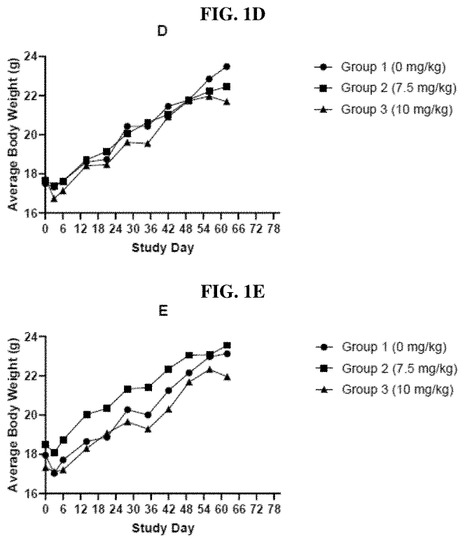

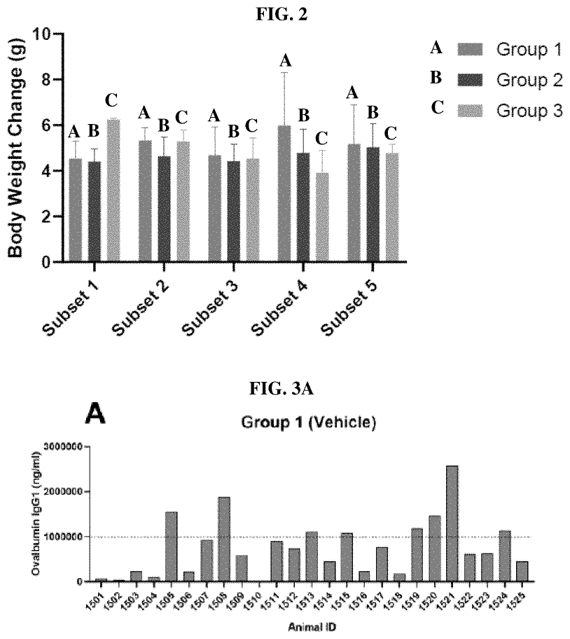

BODY WEIGHT RESULTS: Body weight averages as shown in FIGS. 1B-1F were evaluated for three dose groups (0 mg/kg, 7.5 mg/kg, and 10 mg/kg; dosed IP, twice in one day, 3 hours apart) following immunization on Day 1 with Ovalbumin in CFA. The dose groups were divided into five subgroups (1-5), with the test article being administered on Day 1 (1 hour following immunization; no 10 mg/kg group in FIG. 1A), 3, 6, 14, or 28, respectively. As shown in FIGS. 1A1F, there were no body weight trends attributed to test article administration. FIG. 2 illustrates the average body weight change between Day 1 and D62 among Groups 1-3 and the subgroups thereof (as described above in Table 4). As shown in FIGS. 1-2, there were no body weight trends attributed to test article administration. Generally, all body weights increased between Day 1 and Day 62, with a slight decreased in the mean body weights for all groups following Day 1 immunization.

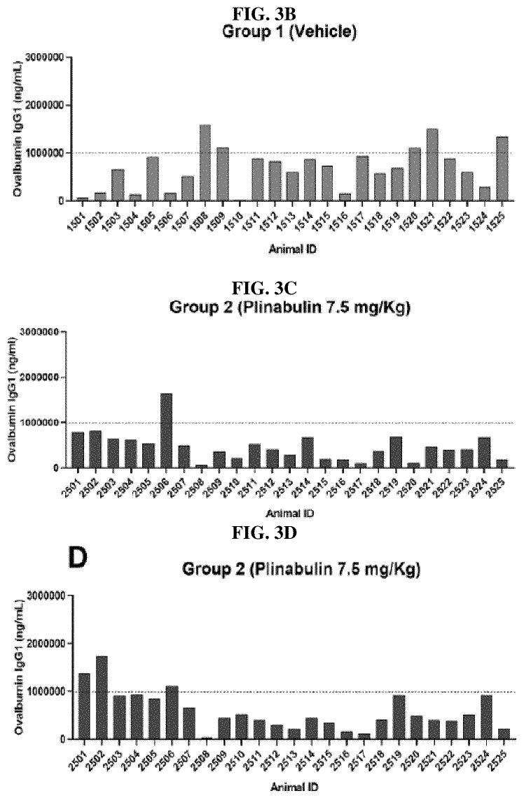

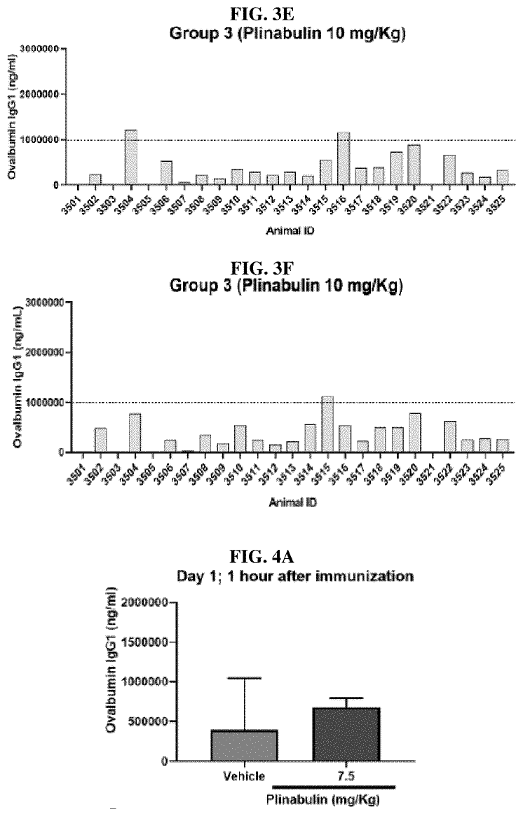

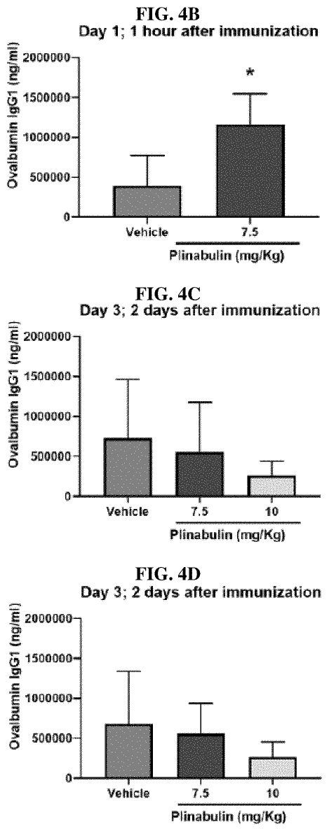

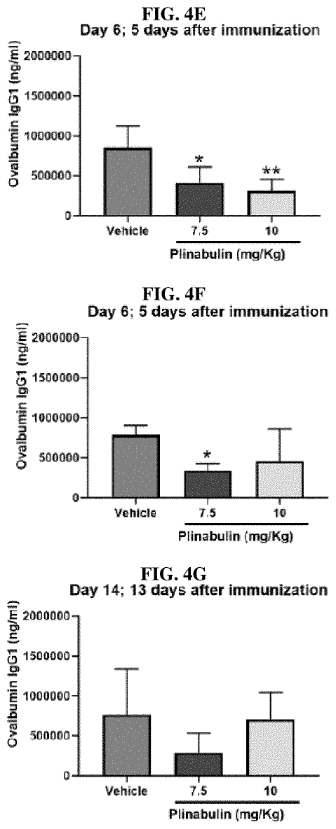

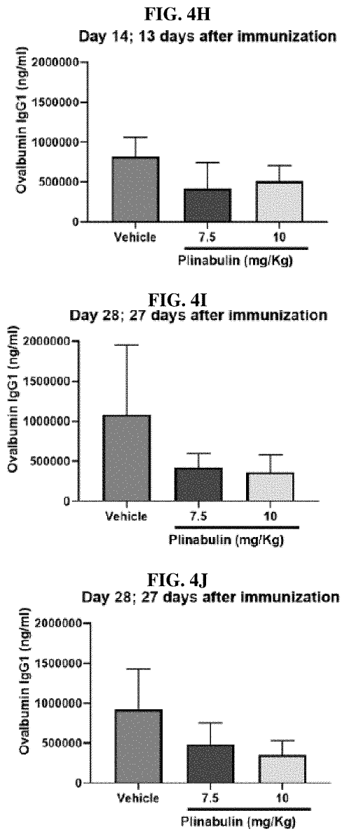

RESULTS OF IGG ANTIBODY ANTI-OVA LEVEL IN SERUM: Serum was evaluated for ovalbumin IgG1 concentration on Day 30 and 62 after subcutaneous immunization of mice with ovalbumin (OVA) in complete Freund's adjuvant (CFA). The concentration of OVA IgG1 in mouse serum was detected by ELISA kit. The concentration of OVA IgG1 in serum from Day 30 was averaged from the data of 1:2,000, 1:6,000 and 1:20,000 dilution (excluded the data out of the standard range). The concentration of OVA IgG1 in serum from Day 62 was from the results of 1:20,000 dilution alone. High variation of serum OVA IgG1 level was found in vehicle group on Day 30 and 62, respectively, in FIGS. 3A-3F. In individual subgroups, Plinabulin at 7.5 and 10 mg/Kg showed inhibitory effects on anti-OVA in 4 out of 5 subgroups (FIG. 4C-4J), on both Day 30 and 62 samples. In subgroup 1 with Plinabulin administrated 1 hour after immunization, Plinabulin dose-dependently increased the production of OVA IgG1 on Day 30 without reaching statistical significance (FIG. 4A). Plinabulin significantly increased OVA IgG1 production on Day 62 at the dose of 7.5 mg/kg in subgroup 1 (FIG. 4B). As shown in FIGS. 3-4, there was a trend for increased anti-OVA response when plinabulin was administered 1 hour after Ovalbumin immunization that reached significance on Day 62 after immunization at 7.5 mg/kg, IP BID (3 hours apart); this group had the highest average anti-OVA IgG1 concentrations of any group in the study. When plinabulin was administered BID for a single day, 3, 6, 14 or 28 days after immunization, anti-OVA IgG1 concentrations were significantly reduced, or tended to be reduced by plinabulin treatment.

### Example 3

### Tubulin Binding Agent Enhances T-Cell Response Elicited by Dendritic Cells

Human peripheral CD14 positive monocytes were collected from a human donor and, subsequently, differentiated and matured into CD14⁺ dendritic cells (DCs). Human CD4 positive T-cells were separately collected from another human donor. The collected CD4⁺ T cells were combined with the CD14⁺ DCs in a mixed lymphocyte reaction (MLR). In this Example, a tubulin binding agent was added, respectively, in the step of monocyte differentiation, in the step of dendritic cell maturation, and in the step of T-cell activation by combining the CD14⁺ DCs with the CD4⁺ T cells.

REAGENTS AND EQUIPMENT USED: Corning® 96-well Clear Flat Bottom Polystyrene TC-treated Microplates (Corning Inc., USA); Corning® 96-well Clear Round Bottom TC-treated Microplate (Corning Inc., USA); Nunc™ EasYFlask™ 25 cm² cell culture flask (Thermo Scientific, USA); RPMI1640 medium (Gibco Co., USA); FBS (Gibco Co., USA); DMSO (Sigma-Aldrich, USA); ACCUSPIN™ System-Histopaque®-1077 (Sigma-Aldrich, USA); IFN-γ ELISA kit (R&D Systems, USA); IL-2 ELISA kit (R&D Systems, USA); LS column (Miltenyi Biotec, USA); CD4⁺ T cell Isolation Kit (Miltenyi Biotec, USA); CD14 Microbeads (Miltenyi Biotec, USA); ImmunoCult™ Dendritic Cell Culture Kit (STEMCELL Technologies, USA); FACS Buffer: PBS+2% FBS; EnVision Multi Label Reader 2104-0010A (PerkinElmer, USA); CO₂ Water Jacketed Incubator (SANYO, Japan); Chongguang XDS-1B reverse microscope (Chongqing Guangdian Corp., China); Eppendorf® Centrifuge (Sigma-Aldrich, USA); and SpectraMax Plus 96-well microplate reader (Molecular Devices Corp., USA).

ISOLATION OF HUMAN PBMCs FROM A DONOR: Peripheral blood mononuclear cells (PBMCs) were isolated from human whole blood according to steps (1a)-(1d) as follows: (1a) Histopaque-1077 was pipetted in a sterile 50-mL centrifuge tube; and an equal volume of the whole blood was carefully layered over the Histopaque-1077 without agitation of the blood-Ficoll interface. (1b) The tube was then centrifuged at 400×g for 30 minutes. The plasma layer on the top was aspirated; and the white translucent interlayer (containing PBMCs) was carefully transferred to a new sterile centrifuge tube. (1c) The obtained mononuclear cells were washed for 2 to 3 times with serum-free RPMI1640 medium; and the tube was spun down at 250×g for 10 min. (1d) The PBMC cell pellets were re-suspended in RPMI1640 medium.

ISOLATION OF CD14⁺ MONOCYTES FROM PBMCs: On Day 1 of the study, PBMC cells were obtained from a human donor according to (1a)-(1d) above. Monocytes were then isolated from these PBMCs on the same day (Day 1) according to steps (2a)-(2k) as follows: (2a) The number of the PBMC cells was determined. (2b) The cell suspension was centrifuged at 300×g for 10 minutes; and the supernatant was aspirated completely. (2c) The cell pellet was re-suspended in 80 μL of FACS buffer per 10⁷ total cells. (2d) Per 10⁷ total cells, 20 μL of CD14 MicroBeads were added. (2e) Mix well and incubate for 15 minutes in the refrigerator (2 to 8° C.). (2f) The cells were washed by adding 1 to 2 mL of FACS buffer per 10⁷ cells and centrifuged at ×1500 rpm for 10 min. (2g) The cells were re-suspended up to 10⁸ cells in 500 μL of FACS buffer. (2h) The column was placed in magnetic field of a suitable MACS Separator. (2i) The column was prepared by rinsing with 3 mL of FACS buffer. (2j) The cell suspension was applied onto the column. Unlabeled cells that pass through were collected and the column was washed for three times each with 3 mL of FACS buffer. Total effluent, which was the unlabeled cell fraction, was collected. Washing steps were performed by adding FACS buffer three times. New buffer was added only when the column reservoir became empty. (2k) The column was then removed from the separator and placed on a suitable collection tube. 5 mL of FACS buffer was pipetted onto the column. The magnetically labeled cells were immediately flushed out by firmly pushing the plunger into the column. This fraction represented the CD14 positive monocytes.

DIFFERENTIATION OF MONOCYTES INTO CD14⁺ DENDRITIC CELLS: From the CD14 positive monocytes obtained from (2k), dendritic cells (DCs) were differentiated according to steps (2l)-(2p) as follows: (21) 5×10⁶ cells were re-suspended per 5 mL of ImmunoCult™ DC Differentiation Medium and mixed well. Then 5 mL of the cell suspension was added to a T-25 cm² flask and the cells were incubated for 3 days at 37° C. and 5% CO₂. (2m) Meanwhile, in parallel, the monocytes were treated with 3, 1, 0.3, 0.1, 0.01 and 0 μM plinabulin, respectively, in the wells of a 6-well plate and incubated for 3 days. (2n) On Day 4, the medium was removed from the T-25 cm² flask by pipetting and added to a 14-mL centrifuge tube. 5 mL of fresh ImmunoCult™ DC Differentiation Medium was quickly added to the culture flask. (2o) The 14 mL tube containing medium and cells (from step (2n)) was centrifuged at 300×g for 10 min. The supernatant was removed and discarded. Cells were resuspended in a small volume (i.e. 50 μL or up to 10% of the original volume) of fresh ImmunoCult™ DC Differentiation Medium and returned to the culture flask in order to save non-adherent or loosely adherent cells. The cells were then incubated at 37° C. for 2 days. (2p) Also on Day 4, 90% differentiation medium for the DCs were removed from the 6-well plate, and 90% fresh differentiation medium were added. The cells were treated with 3, 1, 0.3, 0.1, 0.01 and 0 μM plinabulin as stated above in (2m) (Study Arm #1). After another 2 days incubation, the dendritic cell maturation was evaluated by FACS for CD40, CD80, MHCII and CD86. The rest cells continued without treatment to steps (4a)-(4d) below for MLR assay.

MATURATION OF CD14⁺ DENDRITIC CELLS: The differentiated dendritic cells from step (2p) above were matured according to steps (2q)-(2r) as follows: (2q) On Day 6, ImmunoCult™ Dendritic Cell Maturation Supplement was added directly to the culture flask at a 1-in-100 dilution, for example, 50 μL Maturation Supplement was added to approximately 5 mL culture medium. The culture flask was then swirled gently to mix. The medium was not changed at this point. (2r) Separately, a portion of the differentiated DCs from step (2o) above were treated with 3, 1, 0.3, 0.1, 0.01 and 0 μM plinabulin, respectively, in the wells of a 6-well plate (Study Arm #2). All the dendritic cells were incubated for 2 days for FACS evaluation of maturation markers: CD40, CD80, MHCII and CD86. The rest cells continued to steps (4a)-(4d) below for MLR assay.

ISOLATION OF HUMAN CD4⁺ T CELLS FROM ANOTHER DONOR'S PBMCs: PBMC cells were obtained from another human donor according to (1a)-(1d) above. CD4⁺ T-cells were then isolated from these PBMCs according to steps (3a)-(3k) as follows: (3a) The number of the PBMC cells was determined. (3b) The cell suspension was centrifuged at 300×g for 10 min. The supernatant was then completely aspirated. (3c) The cell pellet was re-suspended in 40 μL of FACS buffer per 10⁷ total cells. (3d) 10 μL of CD4⁺ T Cell Biotin-Antibody Cocktail was added per 10⁷ total cells. (3e) The mixture was well mixed and incubated for 5 min in refrigerator (at 2-8° C.). (3f) 30 μL of FACS buffer was added per 10⁷ total cells, and 20 μL of CD4⁺ T-Cell MicroBead Cocktail was added per 10⁷ total cells. (3g) The mixture was well mixed and incubated for 10 min in refrigerator (at 2 to 8° C.). (3h) A column is placed in magnetic field of a suitable MACS Separator. (3i) The column is prepared by rinsing with 3 mL of buffer. (3j) The cell suspension was applied onto the column. The flow-through containing unlabeled cells, representing the enriched CD4⁺ T cells, were collected. (3k) The column was washed with 3 mL of FACS buffer. Unlabeled cells that passed through, representing the enriched CD4⁺ T cells, were collected and combined with the effluent from step (3j) above.

ALLOGENEIC ACTIVATION OF T CELLS ASSESSED BY MLR ASSAY: Allogeneic Mixed Lymphocyte Reaction (MLR) assays were performed according to steps (4a)-(4d) as follows: (4a) The test article (e.g., plinabulin and other tubulin binding agents) were each diluted in RPMI 1640 medium according to one of Tables 7-9 and, subsequently, added at 50 μL/well in appropriate wells. A duplicate was performed for each condition. (4b) The concentration of the CD4⁺ T-cells obtained from (3k) above was adjusted to 1×10⁶/mL; and, to individual wells of a 96-well plate, 100 μL of the CD4⁺ T-cells was added at 1×10⁵/well. (4c) The DC cells obtained from (2q)-(2r) above were released by adding 2 mM EDTA, collected and centrifuged at ×1500 rpm for 5 min. The concentration of the DC cells was adjusted to 2×10⁵/mL; and 50 μL of the DC cells was added to each of the wells (1×10⁴/well) to obtain a 10:1 ratio of T-cells to dendritic cells. (4d) The plate was incubated at 37° C. for 5 days.

On Day 13, (4e) the cell supernatant was collected for IL-2 and IFN-γ detection by ELISA.

A representative experimental timeline is illustrated in Table 5; and the three study arms in each study of Example 3 are described in Table 6.

TABLE 5
Exemplary Timeline of Cell Preparations and MLR Assay

| Day(s) | Step(s) | Description of the Process |
|---|---|---|
| 1 | (1a)-(1d) | Human PBMCs isolated from a donor |
| 1 | (2a)-(2k) | Human CD14⁺ monocytes isolated from PBMCs |
| 1-5 | (2l)-(2p) | Human CD14⁺ monocytes differentiated into DCs |
| 6-7 | (2q)-(2r) | Human CD14⁺ DCs matured |
| 8 | (1a)-(1d) | Human PBMCs isolated from a different donor |
| 8 | (3a)-(3k) | Human CD4⁺ T cells isolated |
| 8 | (4a)-(4d) | Allogeneic mixed lymphocyte reaction (MLR) assay began |
| 13 | (4e) | Cell supernatant collected for ELISA detection of IL-2 and IFN-γ |

TABLE 6
Study Arm Description

| Study | DC Differentiation | DC Maturation | MLR Assay |
|---|---|---|---|
| Arm | Steps | Steps | Steps |
| #1 | Isolated CD14⁺ | No further | No further |
| | monocytes | plinabulin | plinabulin |
| | treated with | treatment | treatment |
| | plinabulin | | |
| | during DC | | |
| | differentiation | | |
| | (steps (2m) | | |
| | and (2p)) | | |
| #2 | No plinabulin | Differentiated | No further |
| | treatment | DCs treated | plinabulin |
| | | with plinabulin | treatment |
| | | during DC | |
| | | maturation | |
| | | (step (2r)) | |
| #3 | No plinabulin | No plinabulin | Harvested DCs |
| | treatment | treatment | treated with |
| | | | plinabulin (or |
| | | | other agent) |
| | | | in MLR |
| | | | assay |
| | | | (step (4a)) |

### Example 3A

Human PBMCs were isolated from a first donor according to steps (1a)-(1d) as described above in Example 3. Human CD14⁺ monocytes were isolated from the PBMCs according to steps (2a)-(2k), differentiated into human CD14⁺ dendritic cells according to steps (2l)-(2p) and, subsequently, matured according to steps (2q)-(2r) as described above in Example 3. Human PBMCs were isolated from a second donor according to steps (1a)-(1d) and further isolated to obtain human CD4⁺ T cells according to steps (3a)-(3k) as described above in Example 3. The MLR were performed according to steps (4a)-(4d) as described above in Example 3; and Table 7 summarizes the agents tested: plinabulin, nivolumab, and IgG control (and concentrations thereof). Cell supernatant was collected for ELISA measurements of IL-2 and IFN-γ according to step (4e) as described above in Example 3.

TABLE 7
Agents tested in MLR assay

| | | | | | | |
|---|---|---|---|---|---|---|
| Plinabulin (μM) | 3 | 1 | 0.3 | 0.1 | 0.01 | 0 |
| Nivolumab (ng/mL) | 2,000 | 200 | 20 | 2 | 0.2 | 0.02 |
| IgG control (ng/mL) | 2,000 | 200 | 20 | 2 | 0.2 | 0.02 |

Three study arms were: #A1 (tubulin-binding agent treated at step (2m) of DC differentiation), #A2 (tubulin-binding agent treated at step (2r) of DC maturation), and #A3 (tubulin-binding agent treated at step (4a) when the CD4⁺ T cells were combined with the CD14⁺ dendritic cells), as described above in Table 6.

RESULTS:

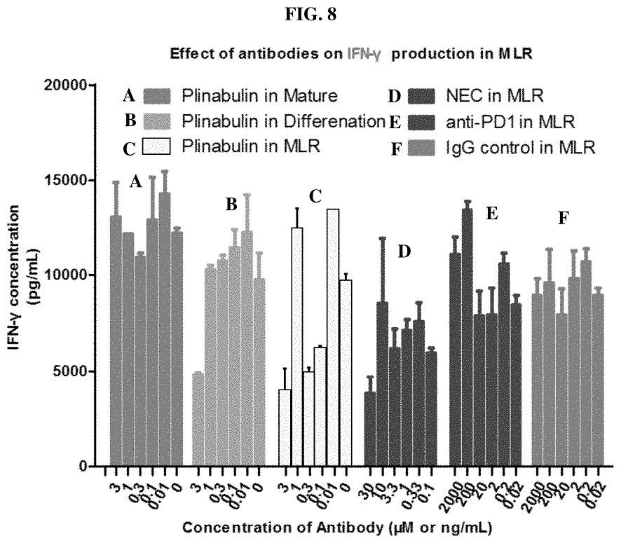

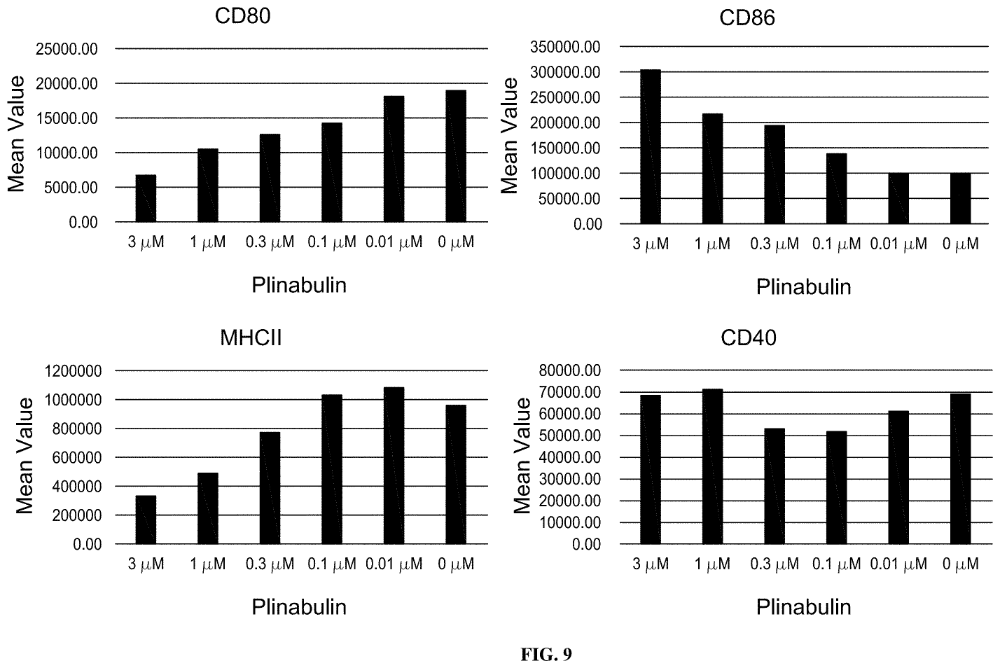

No notable effect of tubulin-binding agent treatment on IFN-γ secretion was observed as shown in FIG. 8.

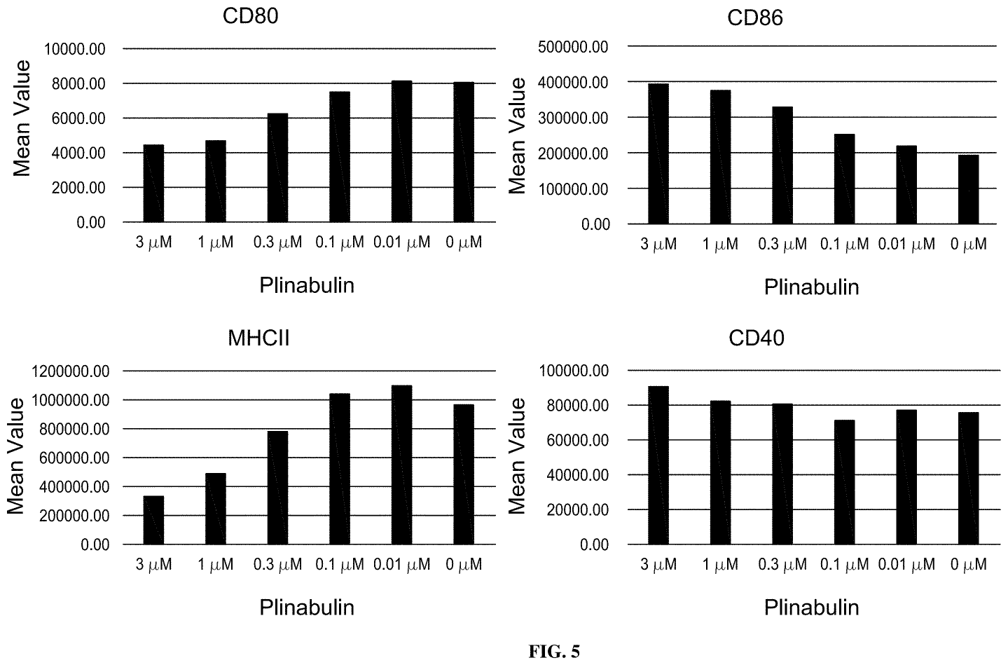

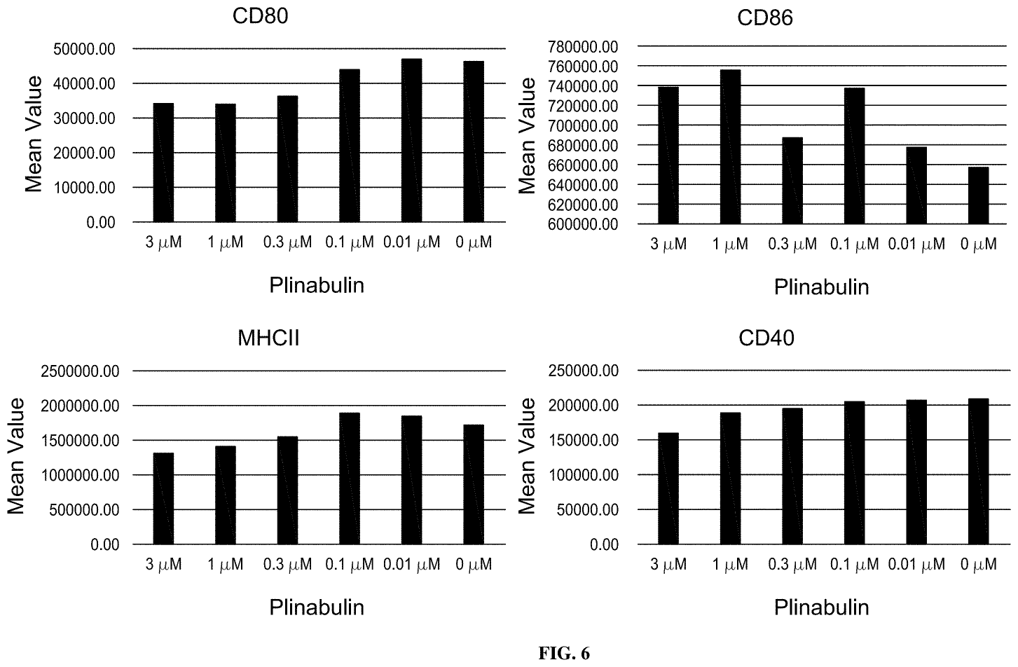

Plinabulin increased CD86 expression on DCs, when the cells were treated either during differentiation from CD14 cells (FIG. 5, upper right) or during DC maturation (FIG. 6, upper right); no discernible increase in MHCII, CD80 and CD40 expression were observed.

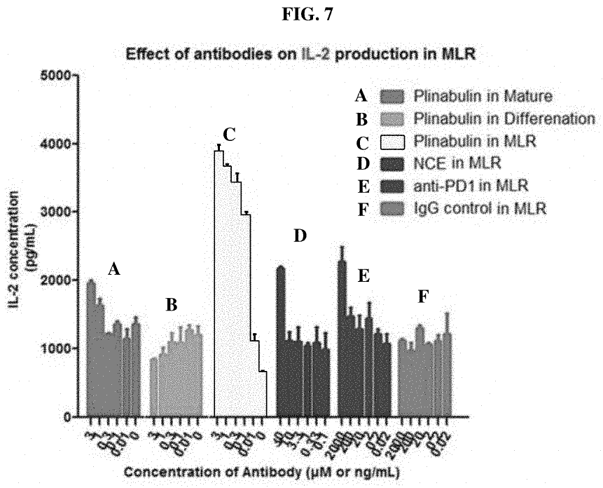

FIG. 7 shows, in the MLR assay, an increase in IL2 secretion in conjunction with increased DC CD86 expression when the DCs were treated with plinabulin (at 1 or 3 μM) during DC maturation (only). FIG. 7 shows a decrease in IL2 secretion when the same concentrations of plinabulin were used to treat CD14⁺ monocytes during DC differentiation. FIG. 7 also shows a significant increase in MLR induced IL-2 secretion, greater than that of nivolumab (at 2 mg/ml), when plinabulin treatment (at ≥100 nM) began at the time when mature DCs were combined with CD4 T-cells.

### Example 3B

Human PBMCs were isolated from a first donor according to steps (1a)-(1d) as described above in Example 3. Human CD14⁺ monocytes were isolated from the PBMCs according to steps (2a)-(2k), differentiated into human CD14⁺ dendritic cells according to steps (2l)-(2p) and, subsequently, matured according to steps (2q)-(2r) as described above in Example 3. Human PBMCs were isolated from a second donor according to steps (1a)-(1d) and further isolated to obtain human CD4⁺ T cells according to steps (3a)-(3k) as described above in Example 3. The MLR were performed according to steps (4a)-(4d) as described above in Example 3; and Table 8 summarizes the agents tested: plinabulin, anti-PD-1, IgG control, docetaxel, and colchicine (and concentrations thereof). Cell supernatant was collected for ELISA measurements of IL-2 and IFN-γ according to step (4e) as described above in Example 3.

TABLE 8
Agents tested in MLR assay

| | | | | | | |
|---|---|---|---|---|---|---|
| Plinabulin (μM) | 3 | 1 | 0.3 | 0.1 | 0.01 | 0 |
| Anti-PD-1 (ng/mL) | 20,000 | 2,000 | 200 | 20 | 2 | 0.2 |
| IgG control (ng/mL) | 20,000 | 2,000 | 200 | 20 | 2 | 0.2 |
| Docetaxel (μM) | 3 | 1 | 0.3 | 0.1 | 0.01 | 0 |
| Colchicine (μM) | 3 | 1 | 0.3 | 0.1 | 0.01 | 0 |

Three study arms were: #B1 (tubulin-binding agent treated at step (2m) of DC differentiation), #B2 (tubulin-binding agent treated at step (2r) of DC maturation), and #B3 (tubulin-binding agent treated at step (4a) when the CD4⁺ T cells were combined with the CD14⁺ dendritic cells), as described above in Table 6.

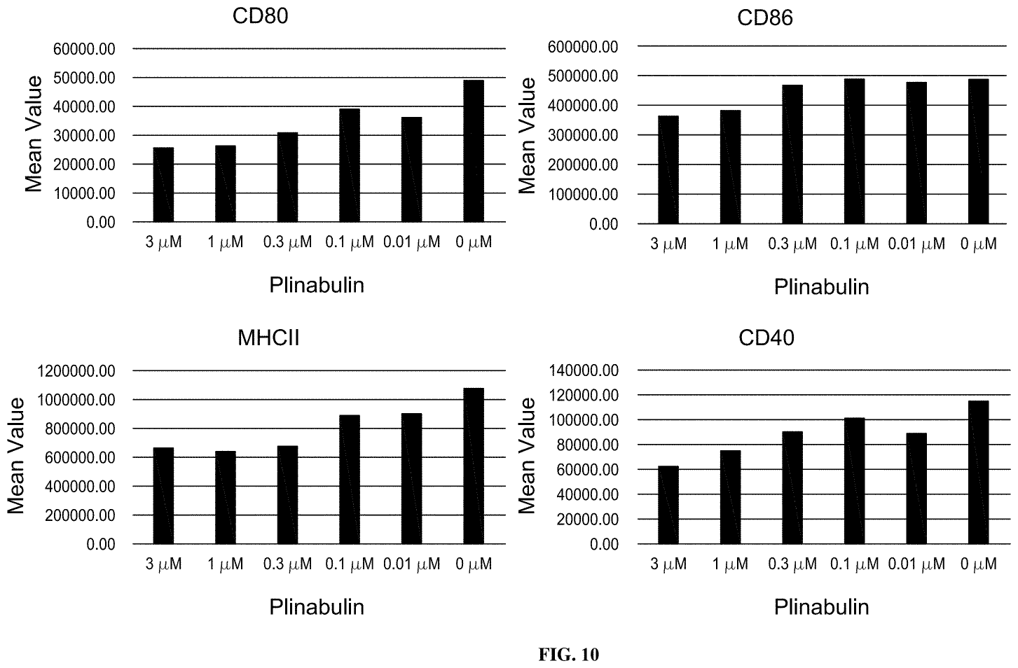

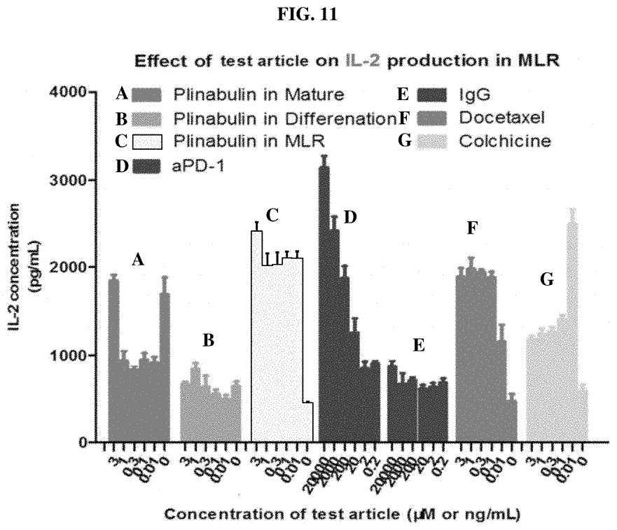

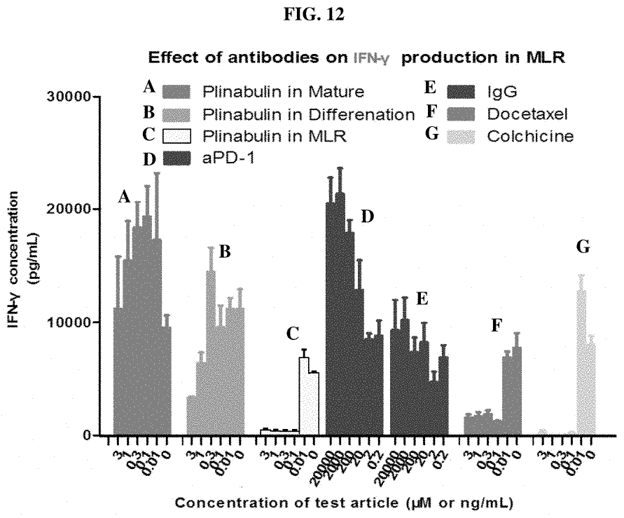

RESULTS: FIGS. 9-10 illustrate the FACS results of Study Arms #B1 and #B2, respectively; and FIGS. 11-12 illustrate the effects of the test article on IL-2 and IFN-y production in MLR, respectively.

### Example 3C

Human PBMCs were isolated from a first donor according to steps (1a)-(1d) as described above in Example 3. Human CD14⁺ monocytes were isolated from the PBMCs according to steps (2a)-(2k), differentiated into human CD14⁺ dendritic cells according to steps (2l), (2n), and (2o) and, subsequently, matured according to step (2q) as described above in Example 3. Steps (2m), (2p) and (2r) were skipped in the study of Example 3C. Human PBMCs were isolated from a second donor according to steps (1a)-(1d) and further isolated to obtain human CD4⁺ T cells according to steps (3a)-(3k) as described above in Example 3. The MLR were performed according to steps (4a)-(4d) as described above in Example 3; and Table 9 summarizes the agents tested: plinabulin, nivolumab, IgG control, fasudil, and colchicine (and concentrations thereof). Cell supernatant was collected for ELISA measurements of IL-2 and IFN-γ according to step (4e) as described above in Example 3.

TABLE 9
Agents tested in MLR assay

| | | | | | | |
|---|---|---|---|---|---|---|
| Plinabulin (nM) | 300 | | | | | |
| Nivolumab (ng/mL) | 20,000 | 2,000 | 200 | 20 | 2 | 0.2 |
| IgG control (ng/mL) | 20,000 | | | | | |
| Fasudil (μM) | 30 | 10 | | | | |
| Colchicine (μM) | 3 | 1 | 0.3 | 0.1 | 0.03 | 0.01 |

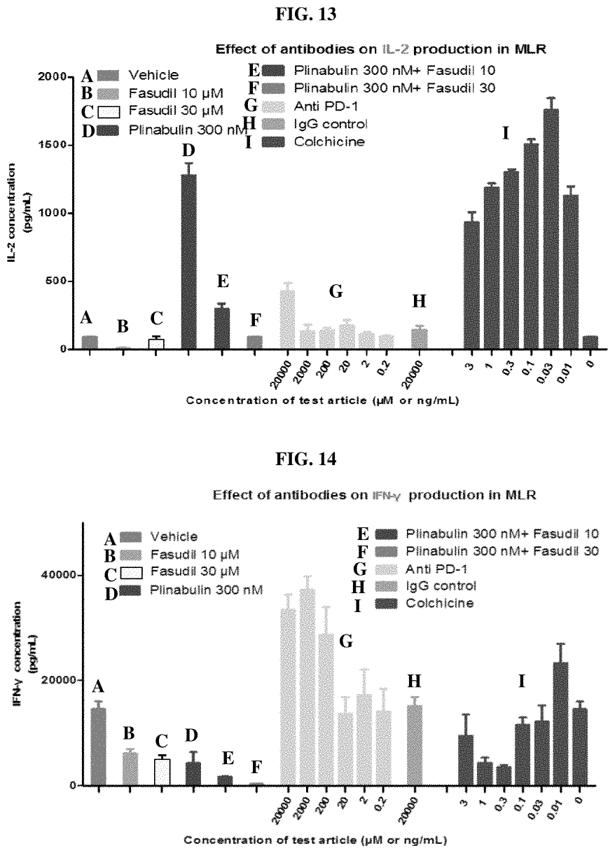

RESULTS: FIGS. 13-14 illustrate the effects of the test article on IL-2 and IFN-γ production in MLR, respectively.

Certain embodiments of the disclosure are encompassed in the claims presented at the end of this specification, or in other claims presented at a later date. Additional embodiments are encompassed in the following set of numbered embodiments:

Embodiment 1. A composition for administration to a subject, comprising a vaccine and a tubulin binding agent.

Embodiment 2. The composition of Embodiment 1, wherein the vaccine comprises an infectious disease vaccine, a cancer vaccine, or a combination thereof.

Embodiment 3. The composition of Embodiment 1 or 2, wherein the vaccine is against one or more infectious diseases selected from the group consisting of cholera, dengue, diphtheria, Haemophilus influzenzae type b (Hib) infection, hepatitis A, hepatitis B, influenza, Japanese encephalitis, meningococcal meningitis, pertussis (aP), polio, rabies, tetanus, tuberculosis (TB), typhoid, and yellow fever (YF), and combinations thereof.

Embodiment 4. The composition of any one of Embodiments 1 to 3, wherein the vaccine is an infectious disease vaccine selected from the group consisting of:
diphtheria and tetanus (DT) vaccine;
diphtheria, tetanus, and pertussis (DTaP) vaccine;
tetanus and diphtheria (Td) vaccine;
tetanus, diphtheria, and pertussis (Tdap) vaccine;
Haemophilus influenzae type b (Hib) conjugate vaccine;
influenza (flu) vaccine;
rabies vaccine;
poliovirus vaccine, such as inactivated poliovirus vaccine (IPV);
meningococcal conjugate vaccine;
typhoid vaccine;
tuberculosis (TB) vaccine; and
yellow fever (YF) vaccine; and
combinations thereof, such as combined DTaP-IPV vaccine and combined DTaP-IPV/Hib vaccine.

Embodiment 5. The composition of any one of Embodiments 1 to 4, wherein the vaccine is selected from the group consisting of:
Haemophilus b Conjugate Vaccine (Tetanus Toxoid Conjugate), such as ActHIB® vaccine or Hiberix® vaccine;
Tetanus Toxoid, Reduced Diphtheria Toxoid and Acellular Pertussis Vaccine Adsorbed, such as Adacel® vaccine;
Diphtheria and Tetanus Toxoids and Acellular Pertussis Vaccine Adsorbed, such as DAPTACEL® vaccine;
Diphtheria and Tetanus Toxoids Adsorbed vaccine;
Influenza Vaccine, such as e.g., Flublok® Quadrivalent vaccine, Fluzone® Quadrivalent vaccine, Fluzone® High-Dose trivalent vaccine, or Fluzone® Intradermal Quadrivalent vaccine;
Human Diploid Cell Rabies Vaccine (HDCV), such as Imovax® vaccine;
Heat-Treated Human Rabies Immune Globulin (HRIG), such as Imogam® vaccine;
Inactivated Poliovirus Vaccine (IPV), such as IPOL® vaccine;
Meningococcal Polysaccharide Diphtheria Toxoid Conjugate Vaccine, such as
Quadrivalent ACYW-135 Menactra® vaccine with diphtheria toxoid carrier;
Diphtheria and Tetanus Toxoids and Acellular Pertussis Adsorbed, Inactivated Poliovirus and Haemophilus b Conjugate, conjugated to tetanus toxoid, Vaccine, such as Pentacel® vaccine;
Diphtheria and Tetanus Toxoids and Acellular Pertussis Absorbed and Inactivated Poliovirus Vaccine, such as Quadracel® vaccine;
Tetanus and Diphtheria Toxoids Adsorbed, such as Tenivac™ vaccine;
Typhoid Vi Polysaccharide Vaccine, such as Typhim Vi® vaccine;
Tuberculin Purified Protein Derivative, such as TUBERSOL® vaccine; and
Yellow Fever Vaccine, such as YF-VAX® vaccine or F-VAX® vaccine.

Embodiment 6. The composition of Embodiment 1 or 2, wherein the vaccine comprises a cancer vaccine.

Embodiment 7. The composition of Embodiment 6, wherein the cancer vaccine comprises an antigen presenting cell (APC)-based vaccine.

Embodiment 8. The composition of Embodiment 6 or 7, wherein the cancer vaccine comprises a dendritic cell (DC)-based vaccine.

Embodiment 9. The composition of any one of Embodiments 6 to 8, wherein the cancer vaccine comprises a B cell-based vaccine.

Embodiment 10. The composition of any one of Embodiments 6 to 9, wherein the cancer vaccine comprises a DNA damaging agent.

Embodiment 11. The composition of any one of Embodiments 1 to 10, wherein the tubulin binding agent functions as a inducer, enhancer or booster of innate or humoral immunity.

Embodiment 12. The composition of any one of Embodiments 1 to 11, wherein the tubulin binding agent is present in an amount effective to stimulate or enhance immune responsiveness in the subject to the vaccine.

Embodiment 13. The composition of any one of Embodiments 1 to 12, wherein tubulin binding agent is plinabulin.

Embodiment 14. The composition of any one of Embodiments 1 to 13, further comprising a pharmaceutically acceptable excipient.

Embodiment 15. The composition of any one of Embodiments 1 to 14, wherein the composition is in a liquid or solid form.

Embodiment 16. The composition of any one of Embodiments 1 to 15, wherein the composition is administered parenterally.

Embodiment 17. The composition of any one of Embodiments 1 to 15, wherein the composition is administered intramuscularly.

Embodiment 18. The composition of any one of Embodiments 1 to 17, wherein the subject is a human.

Embodiment 19. A method of treating or immunizing against a disease, disorder, or condition in a subject, comprising administering to the subject a composition of any one of Embodiments 1 to 18.

Embodiment 20. A method of treating or immunizing against a disease, disorder, or condition in a subject, comprising:
administering to the subject a vaccine; and
administering to the subject a tubulin binding agent.

Embodiment 21. The method of Embodiment 19 or 20, wherein the disease, disorder, or condition is an infectious disease, a cancer, or an immune disorder, or a combination thereof.

Embodiment 22. A method of enhancing an immune response to a vaccine in a subject, comprising:
administering to the subject a vaccine; and
administering to the subject a tubulin binding agent, in an amount sufficient to enhance the immune response to the vaccine as compared to the immune response induced by the vaccine alone.

Embodiment 23. The method of Embodiment 22, wherein said enhancing the immune response comprises inducing lymphocyte cell proliferation; and wherein the lymphocyte cell is a T cell or B cell.

Embodiment 24. The method of Embodiment 23, wherein the lymphocyte cell is a T cell.

Embodiment 25. The method of Embodiment 23, wherein the lymphocyte cell is a CD4⁺ lymphocyte cell.

Embodiment 26. The method of Embodiment 22, wherein said enhancing the immune response comprises inducing B-cell proliferation and differentiation.

Embodiment 27. The method of Embodiment 22, wherein said enhancing the immune response comprises inducing immunoglobulin M (IgM) antibody production, or inducing immunoglobulin G (IgG) antibody production, or a combination thereof.

Embodiment 28. The method of any one of Embodiments 20 to 27, wherein the vaccine is a cancer vaccine or an infectious disease vaccine.

Embodiment 29. The method of any one of Embodiments 20 to 28, wherein the vaccine is selected from the vaccine against one or more diseases selected from the group consisting of cholera, dengue, diphtheria, Haemophilus influzenzae type b infection, hepatitis A, hepatitis B, influenza, Japanese encephalitis, meningococcal meningitis, pertussis, polio, rabies, tetanus, tuberculosis, typhoid, yellow fever, rabies, and tuberculosis.

Embodiment 30. The method of any one of Embodiments 20 to 29, comprising administering the tubulin binding agent and the vaccine simultaneously.

Embodiment 31. The method of any one of Embodiments 20 to 30, comprising administering the tubulin binding agent prior to or after administering the vaccine.

Embodiment 32. The method of any one of Embodiments 20 to 32, wherein the tubulin binding agent is plinabulin.

Embodiment 33. The method of any one of Embodiments 20 to 33, wherein the plinabulin is administered at least about 1 day after the vaccine is administered.

Embodiment 34. The method of any one of Embodiments 20 to 33, wherein the plinabulin is administered at a time between about 2 days and about 6 days after the vaccine is administered.

Embodiment 35. The method of any one of Embodiments 20 to 34, wherein the plinabulin is administered at no greater than 10 mg/kg body weight.

Embodiment 36. The method of Embodiment 35, wherein the plinabulin is administered twice daily about three hours apart.

What is claimed is:

1. A composition for administration to a subject, comprising a vaccine, and a tubulin binding agent.

2. The composition of claim 1, wherein the vaccine is selected from the vaccine against one or more diseases selected from the group consisting of cholera, dengue, diphtheria, Hoemophilus influzenzoe type b infection, hepatitis A, hepatitis B, influenza, Japanese encephalitis, meningococcal meningitis, pertussis, polio, rabies, tetanus, tuberculosis, typhoid, and yellow fever.

3. The composition of claim 1 or 2, wherein the vaccine is selected from the group consisting of ActHIB® Haemophilus b Conjugate Vaccine (Tetanus Toxoid Conjugate); Adacel® Tetanus Toxoid, Reduced Diphtheria Toxoid and Acellular Pertussis Vaccine Adsorbed; DAPTACEL® Diphtheria and Tetanus Toxoids and Acellular Pertussis Vaccine Adsorbed; Diphtheria and Tetanus Toxoids Adsorbed; Flublok®, Quadrivalent Influenza Vaccine; Fluzone® High-Dose Influenza Vaccine; Fluzone® Quadrivalent Influenza Vaccine; Fluzone® Intradermal Quadrivalent Influenza Vaccine; Imovax® Rabies Rabies Vaccine (Human Diploid Cell); IPOL® Poliovirus Vaccine Inactivated; Menactra® Meningococcal (Groups A, C, Y and W-135) Polysaccharide Diphtheria Toxoid Conjugate Vaccine; Pentacel® Diphtheria and Tetanus Toxoids and Acellular Pertussis Adsorbed, Inactivated Poliovirus and Haemophilus b Conjugate (Tetanus Toxoid Conjugate) Vaccine; Quadracel® Diphtheria and Tetanus Toxoids and Acellular Pertussis Absorbed and Inactivated Poliovirus Vaccine; TENIVAC™ Tetanus and Diphtheria Toxoids Adsorbed; Typhim Vi® Typhoid Vi Polysaccharide Vaccine; YF-VAX® Yellow Fever Vaccine; Imogam® Rabies—HT Rabies Immune Globulin (Human) USP, Heat Treated; TUBERSOL® (Tuberculin Purified Protein Derivative, Mantoux); and F-VAX® (Yellow Fever Vaccine).

4. The composition of claim 1 or 2, wherein the tubulin binding agent functions as a inducer, enhancer or booster of innate or humoral immunity.

5. The composition of claim 1, 2, or 4, wherein the vaccine is a vaccine for an infectious disease.

6. The composition of claim 1, 2, or 4, wherein the vaccine is a cancer vaccine.

7. The composition of claim 6, wherein the cancer vaccine comprises an antigen presenting cell based vaccine.

8. The composition of claim 6, wherein the cancer vaccine comprises a dendritic cell based vaccine.

9. The composition of claim 6, wherein the cancer vaccine comprises a B cell based vaccine.

10. The composition of claim 6, wherein the cancer vaccine comprises a DAN damaging agent.

11. The composition of any one of claims 1 to 10, further comprising a pharmaceutically acceptable excipient.

12. The composition of any one of claims 1 to 11, wherein the composition is administered parenterally.

13. The composition of any one of claims 1 to 12, wherein the composition is administered intramuscularly.

14. The composition of any one of claims 1 to 13, wherein the composition is in a liquid or solid form.

15. The composition of any one of claims 1 to 14, wherein the subject is a human.

16. The composition of any one of claims 1 to 15, wherein the amount of the tubulin binding agent is effective to stimulate or enhance immune responsiveness in the subject to the vaccine.

17. The composition of any one of claims 1 to 16, wherein tubulin binding agent is plinabulin.

18. A method of treatment, comprising administering to the subject a vaccine and a tubulin binding agent.

19. A method of enhancing an immune response to a vaccine in a subject, said method comprising administering to the subject a vaccine and a tubulin binding agent , wherein the immune response to the vaccine is enhanced compared to the immune response generated by administration of the vaccine alone to the subject

20. A method of inducing lymphocyte cell proliferation, comprising administering an effective amount of a tubulin binding agent and a vaccine to a subject in need thereof.

21. A method of inducing B cell proliferation, comprising administering an effective amount of a tubulin binding agent and a vaccine to a subject in need thereof.

22. A method of inducing a production of IgM and IgG, comprising administering an effective amount of a tubulin binding agent and a vaccine to a subject in need thereof.

23. A method of enhancing an immune response to a cancer vaccine in a subject, said method comprising administering to the subject a cancer vaccine and a tubulin binding agent, wherein the immune response to the vaccine is enhanced compared to the immune response generated by administration of the cancer vaccine alone to the subject.

24. The method of any one of claims 18 to 22, wherein the vaccine is selected from the vaccine against one or more diseases selected from the group consisting of cholera, dengue, diphtheria, Hoemophilus influzenzoe type b infection, hepatitis A, hepatitis B, influenza, Japanese encephalitis, meningococcal meningitis, pertussis, polio, rabies, tetanus, tuberculosis, typhoid, yellow fever, rabies, and Mycobacterium tuberculosis.

25. The method of any one of claims 18 to 24, comprising administering the tubulin binding agent and the vaccine simultaneously.

26. The method of any one of claims 18 to 25, comprising administering the tubulin binding agent prior to or after administering the vaccine.

27. A method of preparing the composition of claim 1, comprising combining a tubulin binding agent and the vaccine.

28. A method of enhancing an immune response to a vaccine in a subject, said method comprising:
administering to the subject a vaccine, and
administering to the subject a tubulin binding agent after the administration of vaccine, wherein the immune response to the vaccine is enhanced compared to the immune response generated by administration of the vaccine alone to the subject

29. A method of inducing lymphocyte cell proliferation, comprising:
administering to the subject a vaccine, and
administering to the subject a tubulin binding agent after the administration of vaccine.

30. A method of inducing T cell proliferation, comprising:
administering to the subject a vaccine, and
administering to the subject a tubulin binding agent after the administration of vaccine.

31. A method of enhancing an immune response to a cancer vaccine in a subject, said method comprising:
administering to the subject a cancer vaccine, and
administering to the subject a tubulin binding agent after the administration of vaccine, wherein the immune response to the vaccine is enhanced compared to the immune response generated by administration of the cancer vaccine alone to the subject.

32. A method of immunization, comprising:
administering to the subject a vaccine, and administering to the subject a tubulin binding agent after the administration of vaccine.

33. The method of any one of claim 18-32, wherein the tubulin binding agent is plinabulin.

34. The method of any one of claims 18-33, wherein the plinabulin is administered at least about 1 day after the vaccine is administered.

35. The method of any one of claims 18-34, wherein the plinabulin is administered at a time between about 2 days and about 6 days after the vaccine is administered.
